(19) United States
(12) Patent Application Publication
(10) Pub. No.: US 2023/0255483 A1
(43) Pub. Date: Aug. 17, 2023

(54) HYDRATION SENSOR FOR MONITORING AND DIAGNOSIS OF SKIN DISEASES IN ANY ENVIRONMENT AND APPLICATION OF SAME

(71) Applicant: NORTHWESTERN UNIVERSITY, Evanston, IL (US)

(72) Inventors: John A. Rogers, Wilmette, IL (US); Anthony R. Banks, Evanston, IL (US); Kyeongha Kwon, Evanston, IL (US); Jan-kai Chang, Chicago, IL (US)

(21) Appl. No.: 18/009,384

(22) PCT Filed: Jun. 10, 2021
(86) PCT No.: PCT/US2021/036765

§ 371 Date: Dec. 9, 2022

Related U.S. Application Data
(63) Continuation of application No. 17/043,161, filed on Sep. 29, 2020.
(60) Provisional application No. 63/037,092, filed on Jun. 10, 2020.
(60) Provisional application No. 62/650,826, filed on Mar. 30, 2018.
(60) Provisional application No. 62/696,685, filed on Jul. 11, 2018.
(60) Provisional application No. 62/791,390, filed on Jan. 11, 2019.

(51) Int. Cl. A61B 5/00; A61B 5/01

(52) U.S. Cl. CPC A61B 5/0008; A61B 5/01; A61B 5/442; A61B 5/443; A61B 5/445; A61B 2562/0271; A61B 2562/164; A61B 2562/227

(57) ABSTRACT

The invention relates to a hydration sensor comprising a sensing module operably disposed on a target area of interest of skin of a living subject for detecting data associated with thermal properties of the skin; and a wireless platform coupled with the sensing module for wireless data transmission between the sensing module and an external device. The sensing module comprises a thermal actuator for operably heating the target area of interest thereof; and a sensing circuit for simultaneously detecting a transient temperature change thereof to determine thermal properties of the skin.

### CROSS-REFERENCE TO RELATED PATENT APPLICATION

This application claims priority to and the benefit of U.S. Provisional Patent Application No. 63/037,092, filed Jun. 10, 2020, which is incorporated herein in its entirety by reference.

This application is also a continuation-in-part application of U.S. patent application Ser. No. 17/043,161, filed Sep. 29, 2020, which is a national stage entry of PCT Patent Application No. PCT/US2019/025031, filed Mar. 29, 2019, which itself claims priority to and the benefit of U.S. Provisional Patent Application Nos. 62/650,826, filed Mar. 30, 2018, 62/791,390, filed Jan. 11, 2019, and 62/696,685, filed Jul. 11, 2018, which are incorporated herein in their entireties by reference.

### FIELD OF THE INVENTION

The invention relates generally to biosensors, and more particularly, to wireless hydration sensors for rapid, multisensor measurements of hydration levels in healthy and/or diseased skin.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art.

Skin, the largest organ of the human body, is a complex, multilayered functional structure that supports an essential collection of protective, sensory, thermoregulatory, and immunological functions. A core function of skin is to act as a protective interface to the surrounding environment. The three main layers of the skin, stratum corneum (SC), epidermis and dermis, serve as dynamic physical barriers to exogenous insults and active interfaces to maintain homeostasis. Failure of the protective function can result in a range of deleterious health effects, as an impaired skin barrier can lead to infection, insensible water loss, tissue necrosis, and death. Deficiencies in barrier function are also the underlying drivers of atopic dermatitis (AD), commonly known as eczema. AD is the most common inflammatory skin condition, affecting 20% of children and 3% of adults worldwide. Dry skin, or xerosis cutis (XC), is another common skin condition associated with barrier impairment, affecting up to 85% of older adults. Skin barrier dysfunction in neonates can also predict for the development of AD in subsequent years. These and other types of degradation can also increase the systemic absorption of exogenous chemicals and toxic metals, with serious health sequelae.

Quantitative evaluations of skin barrier function can provide essential information to guide clinical decision making. Current methods involve a determination of transepidermal water loss (TEWL) via a measurement of water vapor pressure at the skin surface, or an assessment of the high-frequency electrical properties of the skin itself as a surrogate marker of its water content. Existing TEWL instruments and skin capacitance methods are available only as expensive devices whose accuracy can be influenced by small changes in ambient temperature, by subtle variations in angle and pressure at the skin interface, and by slight user-related differences in testing protocols. Such limitations confine these methods to use in highly controlled clinical and research studies. As an alternative, recent research demonstrates that transient plane source (TPS) methods can be adapted for noninvasive measurements of thermal transport properties of the skin, where simple models quantitatively connect the thermal properties of the skin to its hydration level. Precise, quantitative measurements of the hydration status of skin can yield important insights into dermatological health and skin structure and function, with additional relevance to essential processes of thermoregulation and other features of basic physiology. Existing tools for determining skin water content exploit surrogate electrical assessments performed with bulky, rigid, and expensive instruments that are difficult to use in a repeatable manner.

Therefore, a heretofore unaddressed need exists in the art to address the aforementioned deficiencies and inadequacies.

### SUMMARY OF THE INVENTION

In one aspect, the invention relates to a hydration sensor comprising a sensing module operably disposed on a target area of interest of skin of a living subject for detecting data associated with thermal properties of the skin; and a wireless platform coupled with the sensing module for wireless data transmission between the sensing module and an external device.

In one embodiment, the sensing module comprises a thermal actuator operably disposed on the target area of interest of the skin for heating the target area of interest thereof; and a sensing circuit for simultaneously detecting a transient temperature change (ΔT) thereof to determine thermal properties of the skin.

In one embodiment, the thermal actuator and the sensing circuit are interconnected by serpentine traces to form a flexible structure that facilitates soft, intimate contact to the skin with robust mechanical and thermal coupling.

In one embodiment, the thermal actuator comprises at least one resistor.

In one embodiment, the thermal actuator comprises two or more of surface-mount thin film resistors, thick film resistors, through-hole resistors, and ultrathin-film metal resistors, coupled to each other in series.

In one embodiment, the sensing circuit comprises one or more of negative temperature coefficient thermistors, positive temperature coefficient thermistors, resistance temperature detectors (RTD), and thermocouples.

In one embodiment, the sensing circuit comprises a first pair of negative temperature coefficient thermistors (NTCs) arranged in a first Wheatstone bridge circuit.

In one embodiment, the first pair of NTCs is disposed on a layer different from the thermal actuator, and the first pair of NTCs is directly on the top of the thermal actuator. In another embodiment, the first pair of NTCs is disposed on a layer same as the thermal actuator, and each first NTC has a first distance from the thermal actuator.

In one embodiment, the sensing circuit further comprises a second pair of NTCs arranged in a second Wheatstone bridge circuit serving to compensate for changes in an ambient temperature.

In one embodiment, the second pair of NTCs is disposed on the same layer as the first pair of NTCs, and each second NTC is spatially apart from the first pair of NTCs and has a second distance from the thermal actuator.

In one embodiment, the first and second distances are determined by the design requirement of depth sensitivity into the skin, and ranges from 10s of μm to a few mm.

In one embodiment, the wireless platform comprises at least one of Wi-Fi, BLE, and NFC communication protocols.

In one embodiment, the wireless platform comprises a Bluetooth low energy system on a chip (BLE SoC).

In one embodiment, the BLE SoC comprises a general-purpose input/output (GPIO) electrically coupled to the thermal actuator for providing a periodic current to activate the thermal actuator; a differential amplifier (AMP) electrically coupled to the sensing circuit for amplifying a difference of bridge voltages; an analog-to-digital converter (ADC) electrically coupled to the AMP to digitize output voltages of the AMP; and a BLE radio configured to wirelessly transmit output signals of the ADC to the external device for processing to determine the hydration status of the skin, and receive data from the external device to activate a GPIO pin to provide the periodic current to the thermal actuator.

In one embodiment, a digital on/off switch controlled through a custom application on the external device is adapted to enable BLE-connection and activation of the GPIO pin to source the periodic current into the thermal actuator.

In one embodiment, the BLE SoC further comprises a microcontroller (μC) configured to activate the GPIO pin to source the periodic current into the thermal actuator.

In one embodiment, the hydration sensor further comprises a power module for providing power to the sensing circuit and the wireless platform.

In one embodiment, the power module comprises a battery.

In one embodiment, the battery is a rechargeable battery operably rechargeable with wireless recharging.

In one embodiment, the power module further comprises a wireless charging module for wirelessly charging the rechargeable battery.

In one embodiment, the power module further comprises a failure prevention element including a short-circuit protection component or a circuit to avoid battery malfunction.

In one embodiment, the hydration sensor further comprises a flexible substrate in the form of a flexible printed circuit board (fPCB) with circuit traces that interconnect the thermal actuator on a skin side, the NTCs on an air side, and the BLE SoC.

In one embodiment, the flexible substrate is formed of a flexible material comprising polyimide (PI) and/or polyethylene terephthalate (PET).

In one embodiment, the flexible substrate is a flexible copper-clad polyimide (Cu/PI/Cu) sheet.

In one embodiment, the hydration sensor further comprises an encapsulating enclosure enclosing the thermal actuator, the wireless platform, the battery, and the fPCB.

In one embodiment, the encapsulating enclosure comprises a top layer for thermal, chemical and mechanical isolation of the hydration sensor from the environment; and a bottom layer for providing a direct interface between the thermal actuator at the skin side of the fPCB and the skin.

In one embodiment, the top layer is a shell-like top encapsulation layer including small air gaps for thermally, mechanically, and chemically insulating the critical sensing components.

In one embodiment, the top layer is formed of a flexible material including silicone or silicone gel, low/high density polyethylene (LDPE/HDPE), polystyrene, Teflon®, and various other flexible polymers.

In one embodiment, the bottom layer comprises a flexible adhesive for attaching the hydration sensor to the skin.

In one embodiment, the bottom layer further comprises an ultrathin fabric of fiberglass/reinforcement material embedded in the flexible adhesive layer for enhancing the mechanical robustness of the hydration sensor.

In one embodiment, the reinforcement material is flexible and has varying mesh density and thickness to lend tear resistance to the bottom layer.

In one embodiment, the flexible adhesive layer is formed of silicone or silicone gel, or double-sided skin-safe adhesives, with the ratio of silicone and silicone gel being adjusted to co-optimize mechanical integrity and tackiness of the adhesive.

In one embodiment, the external device is a smartphone, a tablet, a computer, or any electronic device with data reading/processing capability.

In one embodiment, the thermal properties of the skin comprise thermal conductivity and thermal diffusivity of the skin that are related to water content of the skin, wherein the water content is a function of a skin depth.

In one embodiment, the water content is determined from the measured temperature change ΔT vs. time t.

In one embodiment, the water content and skin surface temperature are used to determine a normal state or a disease state of the skin.

In one embodiment, the water content and skin surface temperature serve as quantitative metrics of an efficacy of a treatment of a skin disease, or other health and wellness products including skin moisturizers, lotions, and/or creams.

In one embodiment, the hydration sensor is usable for monitoring the skin condition in a clinical setting and/or an at-home setting.

In one embodiment, the hydration sensor is usable for delivering treatment, monitoring the effects, modulating the treatment protocol as necessary, and/or potentially predicting for flares based on quantitative, individualized measurements on specific lesion sites.

In one embodiment, the hydration sensor is usable for monitoring water content of internal organs for various diseases where traditional monitoring techniques fail to offer continuous assessment of organ health.

In one embodiment, the hydration sensor is usable for monitoring organs during organ transport for applications in organ transplant.

In one embodiment, the hydration sensor is usable for applications to measure thermal conductivity, thermal diffusivity, heat capacity and other thermal properties of any material as a function of depth.

In one embodiment, the hydration sensor is usable for applications to measure water content of any material surface as a function of depth, including hydrogels, plants (irrigation and agriculture applications), food preservation (dried food products, grains, fruits, meats), and/or concrete (industrial applications).

In one embodiment, the hydration sensor is usable for monitoring composition of food/beverages, medicines/industrial chemicals.

In one embodiment, the hydration sensor is re-usable and removal without irritation to the skin or damage to the hydration sensor.

In one embodiment, the hydration sensor is compatible with alcohol-based cleaning wipes allowing for re-use across different users, without any damage to the hydration sensor or loss in efficacy of the hydration sensor adhesive.

In one embodiment, the hydration sensor is sterilizable using alcohol, autoclave steam sterilization, and gas phase sterilization.

In another aspect, the invention relates to a method of fabricating a hydration sensor. In one embodiment, the method includes forming a flexible printed circuit board (fPCB) that interconnects electronics of the hydration sensor; and forming an encapsulating enclosure enclosing the sensing module, the wireless platform and the fPCB, wherein the encapsulating enclosure comprises a top layer and a bottom layer.

In one embodiment, the fPCB is formed of a flexible material comprising polyimide (PI), polyethylene terephthalate (PET), or any one of them in combination with stiff PCB material including FR-4.

In one embodiment, the bottom layer comprises a layered structure of a first flexible layer, a second flexible layer, and a fabric of fiberglass/a reinforcement material embedded between the first flexible layer and the second flexible layer.

In one embodiment, each of the first flexible layer and the second flexible layer is formed of silicone or silicone gel, or double-sided skin-safe adhesives, with the ratio of the silicone and silicone gel being adjusted to co-optimize mechanical integrity and tackiness of the adhesive.

In one embodiment, the reinforcement material is flexible and has varying mesh density and thickness to lend tear resistance to the bottom layer.

In one embodiment, the bottom layer adheres to the f-PCB through use of silicone bonding material, epoxy, glue, or commercial adhesive.

In one embodiment, the top shell layer is formed of silicone or silicone gel, low/high density polyethylene (LDPE/HDPE), polystyrene, Teflon®, and various other flexible polymers.

In one embodiment, the electronics comprises a sensing module for detecting data associated with thermal properties of the skin; and a wireless platform coupled with the sensing module for wireless data transmission between the sensing module and an external device.

In one embodiment, the sensing module comprises a thermal actuator for heating a target area of interest of the skin; and a sensing circuit for simultaneously detecting a transient temperature change (ΔT) thereof to determine thermal properties of the skin.

In one embodiment, the wireless platform comprises at least one of Wi-Fi, BLE, and NFC communication protocols.

In one embodiment, the wireless platform comprises a Bluetooth low energy system on a chip (BLE SoC).

In yet another aspect, the invention relates to a method of monitoring and/or diagnosing a condition of a skin. In one embodiment, the method comprises attaching a hydration sensor onto a target area of interest on the skin, wherein the hydration sensor comprises a thermal actuator, a sensing circuit, and a wireless platform for two-way data communication with an external device; heating the target area of interest of the skin by the thermal actuator, simultaneously detecting data associated with thermal properties of the skin by the sensing circuit, and wirelessly transmitting the detected data, by the wireless platform, to the external device to determiner a transient temperature change (ΔT) thereof; obtaining water content of the target area of interest of the skin from the temperature change (ΔT); and determining a condition of the skin at the target area of interest based on the obtained water content.

In one embodiment, the water content comprises water content ΦE of the epidermis and water content Φ_{D} of the dermis.

In one embodiment, the step of obtaining the water content comprises separately determination of Φ_{E} and Φ_{D} from the temperature change ΔT.

In one embodiment, the wireless platform transmits data through a wireless communication protocol including Near Field Communication (NFC), Wi-fi/Internet, Bluetooth/Bluetooth low energy (BLE), or GSM/Cellular Communication.

In one embodiment, said heating the target area of interest of the skin is formed by providing a periodic current to the thermal actuator.

In one embodiment, activation of the periodic current is controlled by a digital on/off switch through a custom application on the external device.

In one embodiment, said determining the condition of the skin at the target area of interest comprises comparing the obtained water content to a standard water content at the target area of interest so as to determine a normal state or a disease state of the skin.

In one embodiment, said determining the condition of the skin at the target area of interest comprises diagnosing a skin disease at the target area of interest based on wherein the obtained water content thereof.

In one embodiment, said determining the condition of the skin at the target area of interest comprises evaluating an efficacy of a treatment of the skin disease.

In one embodiment, said obtaining water content of the target area of interest of the skin, and said determining a condition of the skin are performed in the external device.

In one embodiment, the method further comprises displaying the condition of the skin at the target area of interest in the external device.

In one embodiment, the method further comprises forwarding the condition of the skin at the target area of interest to a professional and/or a service provider.

In one embodiment, the method further comprises one or more steps of delivering treatment, monitoring the effects, modulating the treatment protocol as necessary, and/or potentially predicting for flares based on quantitative, individualized measurements on specific lesion sites.

In one embodiment, the method is performed under one or more optimized measurement conditions of (1) the measurement being performed rapidly, to minimize effects of occlusion of natural processes of water vapor release from the skin due to the presence of the hydration sensor; (2) very light or zero applied pressure being used during the measurement, to minimize perturbations to the skin; (3) the adhesive being patterned such that it is present only across regions of the hydration sensor device adjacent to the sensor itself, to avoid exfoliation of the skin at the measurement site during peel back, for improved repeatability; (4) the temperature of the hydration sensor being comparable to that of the skin; and (5) skin itself being allowed to acclimate to the surrounding environment prior to the measurement.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIGS. 1A-1F show soft, skin-interfaced platforms for automatic, wireless sensing of thermal transport properties of the skin, according to embodiments of the invention. FIG. 1A: Picture of a thin, flexible thermal actuator/sensor (TAS) module integrated with electronics to provide Bluetooth Low Energy (BLE) communication capabilities, resting on the tip of an index finger. The Inset features an image of the device bent between the thumb and index finger. FIG. 1B: Circuit and block diagrams of the design. The TAS module includes a thermal actuator (Joule heater; R_{H}×2), and Wheatstone bridge circuits that include two thermistors (NTC+, NTC−) with a known resistor (R) on each bridge. A digital on/off switch on the user interface activates a general-purpose input/output (GPIO) pin to source a predetermined periodic current (6.8 mA for 10 s, and 0 mA for 50 s in a 1-min cycle) into the resistive heater. A differential amplifier (AMP) in a BLE system-on-a-chip (SoC) amplifies the difference of the bridge voltages (V+, V−). The subsequent analog-to-digital converter (ADC) samples the AMP output voltages for transmission to a smartphone via BLE radio communication. FIG. 1C: Exploded-view illustration of the constituent layers and components: silicone encapsulation layers, battery, and a flexible copper-clad polyimide (Cu/PI/Cu) sheet with circuit traces that interconnect the thermal actuator (skin side), NTCs (air side), and the BLE SoC. The Inset highlights an air pocket structure defined by the top silicone encapsulation layer as thermal insulation around the TAS module. FIG. 1D: Picture of an encapsulated device adhered to the thenar eminence. FIG. E: Schematic layered view of the TAS module. FIG. F: Schematic top view of the TAS module.

FIGS. 2A-2I show finite-element analysis (FEA) of thermal transport throughout the system as the basis for device optimization and data analysis. FIG. 2A: Schematic illustration of the thermal actuator (R_{H}×2) with two pairs of thermistors: NTC₁ (NTC₁₊, NTC₁₋; resting on the top of the thermal actuator), and NTC₂ (NTC₂₊, NTC₂₋; resting at the same distance, d, from the actuator). FIG. 2B: Schematic illustration of the FEA model of dual-sided (Left) and single-sided (Right) sensor designs. FIG. 2C: FEA results for the temperature distribution of the skin with 50% water by volume, at short (t=1.0 s; Top) and long (t=10 s; Bottom) times after initiating thermal actuation (heating power, Q=20.4 mW). FIGS. 2D-2E: Relationship of ΔT₁₂ at short times (t=1.0 s; FIG. 2D) and long times (t=10 s; FIG. 2E) to epidermis (Φ_{E}) and dermis (Φ_{D}) hydration levels. ΔT₁₂=ΔT₁−ΔT₂. FIGS. 2F-2G: Wireless measurements of ΔT₁₂ at long (FIG. 2F; t=1 to 10 s) and short (FIG. 2G; t=0.5 to 1 s) times for samples that include a thick layer of PDMS S184 (red) and S170 (blue), and a thin layer of S184 with different thickness (70 μm, black; 100 μm, green; 200 μm, yellow) on the top of the S170. FIG. 2H: Comparison between FEA and experiment (SD<3.5%) for PDMS structures described above. FIG. 2I: FEA curve fits of ΔT₁₂ (SD<4.5%) measured for the skin (forearm) throughout the entire measurement period (t=0 to ˜10 s) and the resulting Φ_{D} and Φ_{E}.

FIGS. 3A-3H show experimental studies under various practical conditions. FIG. 3A: Wireless measurements of T₁ (blue) and T₂ (red) in various ambient temperatures (T_{A}) in an oven and a refrigerator (red and blue background, respectively), and at room temperature (RT). FIG. 3B: Measurements of ΔT₁ (blue), ΔT₂ (red), and ΔT₁₂ (black) as a function of T_{A}. FIG. 3C: Wireless measurements of T₁ (blue), T₂ (red), and substrate temperature (T_{S}; green dashed line) on/off the hot plate (heating/cooling, respectively) and with different levels of airflow, as a function of time. The surface temperature of the top encapsulation corresponds to that directly above the heating/sensing elements of the device (TD; purple) and the ambient temperature (T_{A}; black) was determined using a commercial thermometer. FIGS. 3D-3E: Measurements of ΔT₁ (blue), ΔT₂ (red), and ΔT₁₂ (black) as a function of T_{A} (FIG. 3D) and as a function of time (FIG. 3E). A pneumatic flow valve controls the flow of air over the device. FIGS. 3F-3G: Wireless measurements of T₁ (blue), T₂ (red), and the difference (T₁-T₂; black) as a function of time (FIG. 3F), and of ΔT₁ (blue), ΔT₂ (red), and ΔT₁₂ (black) as a function of time (FIG. 3G) underwater. FIG. 3H: Skin hydration levels (Φ) measured by three users at the same set of body locations using the BLE device (Φ_{BLE}), and commercial devices for measuring tissue water content (Φ_{CML,1}) and skin surface hydration levels (Φ_{CML,2}). Five different body locations: forehead (FIG. 3F), right arm (A_{R}), left arm (A_{L}), right leg (L_{R}), and left leg (L_{L}).

FIGS. 4A-4F show experimental studies on the near surface layers of the skin, and on a sample of porcine skin with different, known levels of hydration. FIG. 4A: Optical image of a stripping disk (D-Squame; CuDerm) on the forearm, as a simple and painless means to uniformly remove a fixed area of SC from the skin. FIG. 4B: Measurements of Φ_{BLE} (black) and Φ_{CML,1} (blue), and SC hydration levels (Φ_{CML,3}; red) measured using a commercial device (MoistureMeterSC; Delfin Technologies) as a function of the number of cycles of adhesive disk stripping. FIG. 4C: Measurements of ΔT₁₂ at short (t=1 s; black) and long (t=10 s; red) heating times as a function of the number of cycles of stripping. The vertical bar denotes the spread associated with measurements repeated three times. FIG. 4D: Optical image of the device mounted on a sample of porcine skin, next to a commercial device (MoistureMeterSC; Delfin Technologies) for measuring SC hydration levels. FIG. 4E: Measured Φ for a sample of porcine skin with different, known levels of hydration controlled by placing the sample in a food dehydrator (33° C.). FIG. 4F: Measurements of ΔT₁₂ (square) and linear fits (solid line) at short (t=1 s) and long (t=10 s) heating times for a sample of porcine skin as a function of water loss in grams. The changes in ΔT₁₂ exhibit positive correlation with water loss: ΔT₁₂ (10 s)=6.9+0.3×water loss (R²=0.97), and ΔT₁₂ (1 s)=5.6+0.1×water loss (R²=0.85).

FIGS. 5A-5E show on-body measurements of skin hydration levels. FIGS. 5A-5C: Pictures of devices mounted on the forehead (FIG. 5A), forearm (FIG. 5B), and lower leg (FIG. 5C) of a healthy female volunteer. FIG. 5D: Wireless measurements of ΔT from NTC₁ and NTC₂, and the differences (ΔT₁, ΔT₂, and ΔT₁₂, respectively) acquired from three female (subjects 1, 2, 9; age range: 25 to 27) and seven male (subjects 3 to 8, 10; age range: 17 to 37) healthy volunteers. Mounting positions on the body: forehead (F), right arm (A_{R}), left arm (A_{L}), right leg (L_{R}), and left leg (L_{L}). FIG. 5E: Skin hydration level (Φ) from the values of ΔT₁₂, i.e., Φ_{BLE}, and from a commercial medical device, Φ_{CML,1}. The data exhibit strong correlations between Φ_{BLE} and Φ_{CML,1}:Φ_{CML,1}=Φ_{BLE}×0.80−0.20.

FIGS. 6A-6I show wireless measurements of skin hydration on human subjects with atopic dermatitis. FIGS. 6A-6B: Mounting positions on the back of the hand (atopic eczema) and the forearm (control) of a young adult patient with severe AD (subject 1; FIG. 6A) and on the chest (inflamed, perilesional, and clinically unaffected skin from Left to Right) of an elderly patient with inflammatory AD (subject 2; FIG. 6B). The studies involve three repeated measurements at each location using wireless and commercial devices before and 15 min after application of moisturizer. The Inset shows optical images of the back of the hand, and forearm immediately after application of moisturizer (FIG. 6A) and of devices on the inflamed (Left; FIG. 6A) and perilesional (Right; FIG. 6B) skin. FIGS. 6C-6D: Wireless measurements of ΔT₁₂ before and after (B&A) application of moisturizer from subjects 1 (FIG. 6C) and 2 (FIG. 6D). FIGS. 6E-6F: Skin hydration level (Φ) measured using commercial devices for measuring tissue water content (Φ_{CML,1}) and skin surface (Φ_{CML,2}) hydration, and from the values of ΔT₁₂ (Φ_{BLE}) from subjects 1 (FIG. 6E) and 2 (FIG. 6F). The results of Φ_{CML,1} exhibit strong correlations with Φ_{BLE} after calibration (Φ_{BLE,Cal}=Φ_{BLE}×0.78−0.08). FIGS. 6G-6H: Pictures of the device mounted on the forehead (FIG. 6G) and leg (FIG. 6H; visibly dry skin) of a toddler. FIG. 6I Measurements of Φ_{BLE} (blue), Φ_{CML,1} (black), TEWL (red), and SCH (green) on the left leg (L_{L}), right leg (R_{L}), and forehead (F_{H}). The vertical lines denote the error bars.

FIGS. 7A-7I show studies of the effects of moisturizers on healthy adults—changes in Φ_{CML,1} and Φ_{BLE} from three different skin locations without moisturizer (control; FIGS. 7A, 7D and 7G), and 1 min (short; FIGS. 7B, 7E and 7H) and 15 min (long; FIGS. 7C, 7F and 7I) after application of moisturizer on the forearms of subjects 1 (FIGS. 7A-7C), 2 (FIGS. 7D-7F), and 3 (FIGS. 7G-7I). Measurements are normalized to each initial value determined at time=0.

FIG. 8 shows a picture of an encapsulated device next to a 12 mAh Li-polymer battery.

FIG. 9 shows wireless read-out of the temperature change measured from NTC₁ (ΔT₁) and NTC₂ (ΔT₂) as a function of time for 10 s of heating every one min. ΔT₁₂=ΔT₁−ΔT₂.

FIGS. 10A-10B show schematic illustration of the FEA model of dual-sided (FIG. 10A) and single-sided (FIG. 10B) sensor designs. FIGS. 10C-10D show sensitivities of the temperature difference (ΔT₁₂) between NTC₁ (ΔT₁) and NTC₂ (ΔT₂) to skin hydration level of dual-sided (FIG. 10C) and single-sided (FIG. 10D) designs 10 s after the heater is activated.

FIGS. 11A-11E show comparisons between FEA and measurement for a thick layer of S184 (FIG. 11A) and S170 (FIG. 11B), and a thin layer of S184 (70 μm, FIG. 11C; 100 μm, FIG. 11D; 200 μm, FIG. 11E) on top of the S170.

FIGS. 12A-12B show computational predictions of ΔT as a function of skin hydration level (Φ) with different values of d (FIG. 12A; Q=20.4 mW, t=10.0 s), and ΔT as a function of Q (FIG. 12B; d=1.2 mm, t=10.0 s).

FIGS. 13A-13B show computational predictions of ΔT₁₂ with different sizes of actuators (width and length of RH) for 30% (FIG. 13A) and 95% (FIG. 13B) hydrated skin.

FIGS. 14A-14B show an effect of design parameters on the temperature change. FIG. 14A: A simplified, analytical model of a disk-shaped thermal actuator (radius, R) and NTCs. FIG. 14B: Analytical scaling law for ΔT₁₂.

FIG. 15 shows ambient temperatures. Measurements of ΔT₁ (blue), ΔT₂ (red), and ΔT₁₂ (black) as a function of time (min). The values of ΔT₁ and ΔT₂ fluctuate at the moment the device enters and exits the oven (yellow background).

FIG. 16A shows a picture of conventional devices based on skin capacitance measurements for monitoring tissue water content (MoistureMeterD; top), SC hydration levels (MoistureMeterSC; middle top), and skin surface hydration levels (Gpskin; middle bottom), and a BLE device (bottom). FIG. 16B shows a picture of devices on the forearm, according to embodiments of the invention. The commercial devices require care by the user to hold the probe and apply a certain pressure against the skin for each measurement.

FIG. 17 shows mounting positions on the body: forehead (F), right arm (A_{R}), left arm (A_{L}), right leg (L_{R}), and left leg (L_{L}).

FIG. 18 shows SD for Φ tested by 3 users using BLE (Φ_{BLE}) and commercial (Φ_{CML,1} and Φ_{CML,2}) devices at five different body locations, forehead (F), right arm (A_{R}), left arm (A_{L}), right leg (L_{R}), and left leg (L_{L}), for subject 1 to 3.

FIG. 19 shows a positive correlation between Φ_{BLE} and Φ_{CML,1} (black), and between Φ_{BLE} and Φ_{CML,2} (red), and their linear fits (lines).

FIGS. 20A-20B show bland-Altman plots of Φ_{BLE,Cal1} and Φ_{CML,1} (FIG. 20A), and Φ_{BLE,Cal2} and Φ_{CML,2} (FIG. 20B). Horizontal lines represent the mean (red), and mean±1.96·SD (blue) values of ΦBLE,Cal−Φ_{CML} where SD is the standard deviation. The mean±SD values of the differences (Φ_{CML,1}−Φ_{BLE,Cal1}, and Φ_{CML,2}−Φ_{BLE,Cal2}) are 0.00±0.02 and 0.00±0.04, respectively.

FIG. 21 shows pictures of an encapsulated device mounted on a pediatric hand.

FIG. 22 shows SD for ΔT₁, ΔT₂, and ΔT₁₂ at five different body locations, forehead (F), right arm (A_{R}), left arm (A_{L}), right leg (L_{R}), and left leg (L_{L}), for subjects 1 to 10.

FIG. 23 shows a positive correlation between skin hydration level from wireless (Φ_{BLE}) and commercial (Φ_{CML,1}) devices, and its linear fit (red line). Linear fits indicate that Φ_{CML,1}=Φ_{BLE}×0.80−0.20, with a coefficient of determination of R²=0.66.

FIG. 24 shows a Bland-Altman plot (difference plot) of Φ_{BLE,Cal1} and Φ_{CML,1}. Horizontal lines represent the mean (red; ˜0.00), and mean±1.96·SD (blue; ˜0.00±1.96·0.05) values of Φ_{BLE,Cal1}−Φ_{CML,1} where SD is the standard deviation.

FIG. 25A shows pictures of the device on a subject's leg before (left) and after (right) shaving the skin. Insets show the sensing point. FIG. 25B shows wireless measurements of ΔT₁ (blue), ΔT₂ (red), and ΔT₁₂ (black) before and after shaving the sensing area.

FIG. 26A shows an optical image of the device mounted on the forehead of a healthy male subject. FIG. 26B shows wireless measurements of Φ_{BLE} before, during and after a workout. Vertical bar denotes the error bar over 3-time measurements.

FIG. 27 shows an optical image of the device mounted on the atopic hand of a subject 1, next to a BLE-enabled smartphone.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

“Interfacing” refers to the positioning of the device with tissue such that the device may affect the tissue, and vice versa. For example, a thermal actuator of the device may result in a thermal load provided to the tissue in the form of a “thermal input”. The thermal input is preferable a heating action, although the device is also compatible with a cooling action. “Thermally interfacing”, therefore, refers to the ability of the device to affect a thermal challenge on underlying tissue, and to detect a response thereto, such as a change in temperature over time, including for a time period after the thermal input ends. In this manner, one or more tissue parameters may be determined, such as tissue hydration, inflammation, blood flow, UV damage.

The terms “flexible” and “bendable” are used synonymously in the present description and refer to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. As used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and mesh geometries.

Any of the devices provided herein may be described in terms of elasticity or elastic. “Elasticity” refers to a measure of a non-plastic deformation, such as a deformation that can undergo deformation and relaxation back to the original undeformed, state without substantial creep, including under repeated deformatory stresses and relaxation cycles. The creep may be defined as less than a 5%, less than 2%, or less than 1% permanent deformation or change in the original material property.

“Stretchable” refers to the ability of a material, structure, device or device component to be strained without undergoing fracture. In an exemplary embodiment, a stretchable material, structure, device or device component may undergo strain larger than 0.5% without fracturing, for some applications strain larger than 1% without fracturing and for yet other applications strain larger than 3% without fracturing. As used herein, many stretchable structures are also flexible. Some stretchable structures (e.g., device components) are engineered to be able to undergo compression, elongation and/or twisting so as to be able to deform without fracturing. Stretchable structures include thin film structures comprising stretchable materials, such as elastomers; bent structures capable of elongation, compression and/or twisting motion; and structures having an island—bridge geometry. Stretchable device components include structures having stretchable interconnects, such as stretchable electrical interconnects.

“Two-way communication” refers to the ability to wirelessly communicate with the device, such that power, commands or queries are sent to, and acted on, the device and the device itself can send information or diagnostics to an external controller that is wirelessly connected to the device. Accordingly, an “external controller” refers to an off-board component that can control and received information from the device. Examples include hand-held devices, computers, smartphones, and the like.

The devices and methods provided herein are suited for long-term use in that the device may be “worn” over long periods of time and remain functional. Accordingly, “continuous” refers to the time period any of the devices provided herein are deployed on or in biological tissue and is ready for use. While the device is continuously deployed, the measurement may be described as intermittent or periodic, such as for a continuous measurement time on the order of minutes, such as greater than or equal to 1 minute, 5 minutes, 10 minutes or 20 minutes. The periodic measurement, however, can be repeated over the time period the device is worn, such as in the morning, during the day, and in the evening, including on the order of 12 hours or more, 1 day or more, or 7 days or more.

“Thermal parameter” or “thermal transport property” may refer to a rate of change of a temperature-related tissue property, such as a heat-related tissue property, over time and/or distance (velocity). In some embodiments, the heat-related tissue property may be temperature, conductivity or humidity. The heat-related tissue property may be used to determine a thermal transport property of the tissue, where the “thermal transport property” relates to heat flow or distribution at or near the tissue surface. In some embodiments, thermal transport properties include temperature distribution across a tissue surface, thermal conductivity, thermal diffusivity and heat capacity. Thermal transport properties, as evaluated in the present methods and systems, may be correlated with a physical or physiological property of the tissue. In some embodiments, a thermal transport property may correlate with a temperature of tissue. In some embodiments, a thermal transport property may correlate with a vasculature property, such as blood flow and/or direction.

“Substrate” refers to a portion of the device that provides mechanical support for a component(s) disposed on or within the substrate. The substrate may have at least one skin-related function or purpose. For example, the substrate may have a mechanical functionality, for example, providing physical and mechanical properties for establishing conformal contact at the interface with a tissue, such as skin or a nail surface. The substrate may have a thermal loading or mass small enough so as to avoid interference with measurement and/or characterization of a tissue parameter. The substrate of any of the present devices and methods may be biocompatible and/or bioinert. A substrate may facilitate mechanical, thermal, chemical and/or electrical matching to the underlying tissue, such as skin or nail of a subject such that the mechanical, thermal, chemical and/or electrical properties of the substrate and the tissue are within 20%, or 15%, or 10%, or 5% of one another.

A flexible substrate that is mechanically matched to a tissue, such as skin, provides a conformable interface, for example, useful for establishing conformal contact with the surface of the tissue. Devices and methods described herein may incorporate mechanically functional substrates comprising soft materials, for example exhibiting flexibility and/or stretchability, such as polymeric and/or elastomeric materials. A mechanically matched substrate may have a Young's modulus less than or equal to 100 MPa, and optionally for some embodiments less than or equal to 10 MPa, and optionally for some embodiments, less than or equal to 1 MPa. In an embodiment, a mechanically matched substrate has a thickness less than or equal to 0.5 mm, and optionally for some embodiments, less than or equal to 1 cm, and optionally for some embodiments, less than or equal to 3 mm. In an embodiment, a mechanically matched substrate has a bending stiffness less than or equal to 1 nN m, optionally less than or equal to 0.5 nN m.

In some embodiments, a mechanically matched substrate is characterized by one or more mechanical properties and/or physical properties that are within a specified factor of the same parameter for an epidermal layer of the skin or nail, such as a factor of 10 or a factor of 2. For example, a substrate may have a Young's Modulus or thickness that is within a factor of 20, or optionally for some applications within a factor of 10, or optionally for some applications within a factor of 2, of a tissue, such as an epidermal layer of the skin or of the nail surface, at the interface with a device of the present invention. A mechanically matched substrate may have a mass or modulus that is equal to or lower than that of skin.

In some embodiments, a substrate that is thermally matched to skin has a thermal mass small enough that deployment of the device does not result in a thermal load on the tissue, such as skin, or small enough so as not to impact measurement and/or characterization of a physiological parameter. In some embodiments, for example, a substrate that is thermally matched to skin has a thermal mass low enough such that deployment on skin results in an increase in temperature of less than or equal to 2 degrees Celsius, and optionally for some applications less than or equal to 1 degree Celsius, and optionally for some applications less than or equal to 0.5 degree Celsius, and optionally for some applications less than or equal to 0.1 degree Celsius. In some embodiments, for example, a substrate that is thermally matched to skin has a thermal mass low enough that is does not significantly disrupt water loss from the skin, such as avoiding a change in water loss by a factor of 1.2 or greater. Therefore, the device does not substantially induce sweating or significantly disrupt transdermal water loss from the skin, while maintaining an effectiveness of determining hydration state of the skin.

The substrate may have a Young's modulus less than or equal to 100 MPa, or less than or equal to 50 MPa, or less than or equal to 10 MPa, or less than or equal to 100 kPa, or less than or equal to 80 kPa, or less than or equal to 50 kPa. Further, in some embodiments, the device may have a thickness less than or equal to 5 mm, or less than or equal to 2 mm, or less than or equal to 100 μm, or less than or equal to 50 μm, and a net bending stiffness less than or equal to 1 nN m, or less than or equal to 0.5 nN m, or less than or equal to 0.2 nN m. For example, the device may have a net bending stiffness selected from a range of 0.1 to 1 nN m, or 0.2 to 0.8 nN m, or 0.3 to 0.7 nN m, or 0.4 to 0.6 nN m.

In an embodiment, “epidermal tissue” refers to the outermost layers of the skin or the epidermis. The epidermis is stratified into the following non-limiting layers (beginning with the outermost layer): stratum corneum, stratum lucidum (on the palms and soles, i.e., the palmar regions), stratum granulosum, stratum spinosum, stratum germinativum (also called the statum basale). In an embodiment, epidermal tissue is human epidermal tissue.

“Encapsulate” refers to the orientation of one structure such that it is at least partially, and in some cases completely, surrounded by one or more other structures, such as a substrate, adhesive layer or encapsulating layer. “Partially encapsulated” refers to the orientation of one structure such that it is partially surrounded by one or more other structures, for example, wherein 30%, or optionally 50%, or optionally 90% of the external surface of the structure is surrounded by one or more structures. “Completely encapsulated” refers to the orientation of one structure such that it is completely surrounded by one or more other structures. The encapsulation may be described in functional terms, such as being a fluid or electrical barrier, particularly in those locations where a fluid or electrical field would lead to an adverse impact on the device.

“Conformable” refers to a device, material or substrate which has a bending stiffness that is sufficiently low to allow the device, material or substrate to adopt any desired contour profile, for example a contour profile allowing for conformal contact with a curvilinear surface, including a surface whose shape may change over time, such as with physical exertion or normal every day movement, such as skin.

“Conformal contact” refers to contact established between a device and a receiving surface. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In an embodiment, conformal contact involves adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. Devices of certain aspects are capable of establishing conformal contact with internal and external tissue. Devices of certain aspects are capable of establishing conformal contact with tissue surfaces characterized by a range of surface morphologies including planar, curved, contoured, macro-featured and micro-featured surfaces and any combination of these. Devices of certain aspects are capable of establishing conformal contact with tissue surfaces corresponding to tissue undergoing movement, including an internal organ or skin.

“Young's modulus” is a mechanical property of a material, device or layer which refers to the ratio of stress to strain for a given substance. Young's modulus may be provided by the expression:

$$E = \frac{( stre s s )}{( s t r a i n )} = ( \frac{{L}_{0}}{Δ L} ) ( \frac{F}{A} ) ,$$

where E is Young's modulus, Lo is the equilibrium length, ΔL is the length change under the applied stress, F is the force applied, and A is the area over which the force is applied. Young's modulus may also be expressed in terms of Lame constants via the equation:

$$E = \frac{μ ( 3 λ + 2 μ )}{λ + μ} ,$$

where λ and μ are Lame constants. High Young's modulus (or “high modulus”) and low Young's modulus (or “low modulus”) are relative descriptors of the magnitude of Young's modulus in a given material, layer or device. In some embodiments, a high Young's modulus is larger than a low Young's modulus, preferably about 10 times larger for some applications, more preferably about 100 times larger for other applications, and even more preferably about 1000 times larger for yet other applications. In an embodiment, a low modulus layer has a Young's modulus less than 100 MPa, optionally less than 10 MPa, and optionally a Young's modulus selected from the range of 0.1 MPa to 50 MPa. In an embodiment, a high modulus layer has a Young's modulus greater than 100 MPa, optionally greater than 10 GPa, and optionally a Young's modulus selected from the range of 1 GPa to 100 GPa. In an embodiment, a device of the invention has one or more components having a low Young's modulus. In an embodiment, a device of the invention has an overall low Young's modulus.

“Low modulus” refers to materials having a Young's modulus less than or equal to 10 MPa, less than or equal to 5 MPa or less than or equal to 1 MPa.

Use of the term “effective” with any physical parameter reflects an average or bulk parameter. This reflects, for example, that the devices are not formed of a single unitary material, but can have materials ranging from elastomers, adhesives, thin films, metals, semiconductors, integrated circuits and other materials that span orders of magnitudes. An effective device modulus, accordingly, can reflect physical properties of the entire device, with a special geometry and configuration of components to ensure the bulk behavior of the device is tailored to the application of interest. For skin, the entire device can be configured to be highly flexible and stretchable, with certain portions that are by necessity less flexible and stretchable due to material requirements. For a nail, the entire device need not be so stretchable, but should still conform to the nail curvilinear surface contour.

“Bending stiffness” is a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

“Tissue parameter” refers to a property of a tissue including a physical property, physiological property, electronic property, optical property and/or chemical composition. Tissue parameter may refer to a surface property, a sub-surface property or a property of a material derived from the tissue, such as a biological fluid. Tissue parameter may refer to a parameter corresponding to an in vivo tissue such as temperature; hydration state; chemical composition of the tissue; chemical composition of a fluid from the tissue; pH of a fluid from the tissue; the presence of absence of a biomarker; intensity of electromagnetic radiation exposed to the tissue; wavelength of electromagnetic radiation exposed to the tissue; and amount of an environmental contaminant exposed to the tissue. Devices of some embodiments are capable of generating a response that corresponds to one or more tissue parameters, such as for a low hydration state application of a hydrating material (e.g., a moisturizer), or for a UV damage state application of a UV block (e.g., sunscreen) or a warning to the individual wearing the device, such as a haptic feedback actuator that provides a vibratory signal, optical signal, or electrical signal, warning the user to take appropriate action. A tissue parameter may provide useful information about the health of a tissue. For example, a tissue parameter that is a “sunburn parameter” may be used to assess effectiveness of a compound as a sunscreen, to warn a user, or to automatically apply a treatment, including application of a sunscreen. The sunburn parameter may be an optical property, such as color, or may be a hydration property that, in turn, is related to thermal conductivity of the underlying tissue.

Any of the devices and methods provided herein may be personalized to a user. In this context, “personalized” refers to the device or method that is tailored to that of an individual user, recognizing there may be relatively significant person-to-person variability with respect to one or more baseline tissue parameters, and tissue behavior to a stimulus. For example, some people may have higher inherent thermal conductivity, or high resting hydration level. The devices or methods may accurately determine the baseline tissue parameter, with monitoring and corresponding treatment tailored to that individual's baseline tissue parameter.

“Haptic feedback element” refers to a device component that generates a physically-detectable stimulus by a user, such as be a haptic feedback element that is selected from the group consisting of a vibrator, an optical light source, or an electrode.

“Environmental parameter” refers to a property of an environment of a device, such as a device in conformal contact with a tissue. Environment parameter may refer to a physical property, electronic property, optical property and/or chemical composition, such as an intensity of electromagnetic radiation exposed to the device; wavelengths of electromagnetic radiation exposed to the device; a chemical composition of an environmental component exposed to the device; chemical composition of an environmental component exposed to the device; amount of an environmental contaminant exposed to the device; and/or chemical composition of an environmental contaminant exposed to the device. Devices of some embodiments are capable of generating a response that corresponds to one or more environmental parameters. For example, in low humidity conditions, application of a hydrating material; high UV conditions, application of a UV block material.

“Power harvesting” refers to a process by which energy is derived from an external source and, thereby, may avoid the need for relatively large, bulky and expensive primary or secondary battery systems. Of course, the devices provided herein may be compatible with batteries and/or supercapaciters, depending on the application of interest. For example, relatively heavy or bulky systems may be incorporated into clothing, shoes, hats, gloves, scarves, face masks, and the like, in a manner that would be unobtrusive, or minimally noticeable, to a user.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Precise, quantitative measurements of the hydration status of skin can yield important insights into dermatological health and skin structure and function, with additional relevance to essential processes of thermoregulation and other features of basic physiology. Existing tools for determining skin water content exploit surrogate electrical assessments performed with bulky, rigid, and expensive instruments that are difficult to use in a repeatable manner. Recent alternatives exploit thermal measurements using soft wireless devices that adhere gently and noninvasively to the surface of the skin, but with limited operating range (˜1 cm) and high sensitivity to subtle environmental fluctuations.

Accordingly, this invention, among other things, discloses a set of ideas and technologies that overcome these drawbacks to enable high-speed, robust, long-range automated measurements of thermal transport properties via a miniaturized, multisensor module controlled by a long-range (˜10 m) Bluetooth Low Energy (BLE) system on a chip (SoC), with a graphical user interface to standard smartphones. Soft contact to the surface of the skin, with almost zero user burden, yields recordings that can be quantitatively connected to hydration levels of both the epidermis and dermis, using computational modeling techniques, with high levels of repeatability and insensitivity to ambient fluctuations in temperature. Systematic studies of polymers in layered configurations similar to those of human skin, of porcine skin with known levels of hydration, and of human subjects with benchmarks against clinical devices validate the measurement approach and associated sensor hardware. The results support capabilities in characterizing skin barrier function, assessing severity of skin diseases, and evaluating cosmetic and medication efficacy, for use in the clinic or in the home.

The following exemplary embodiments further illustrate the invention but should not be construed as in any way limiting its scope.

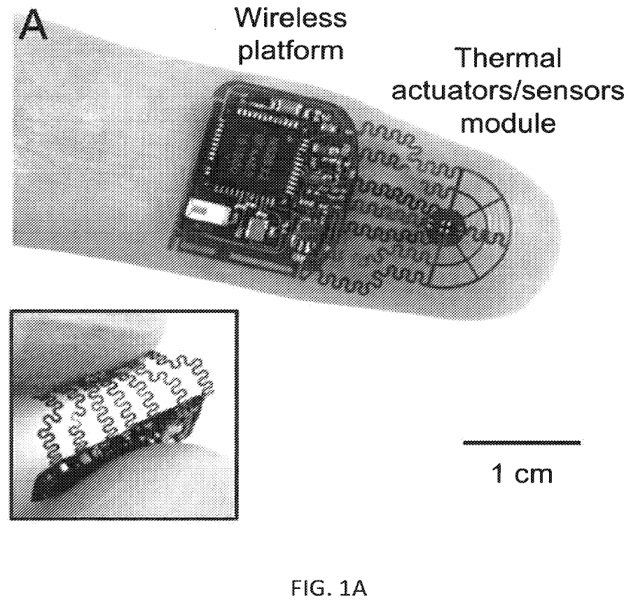

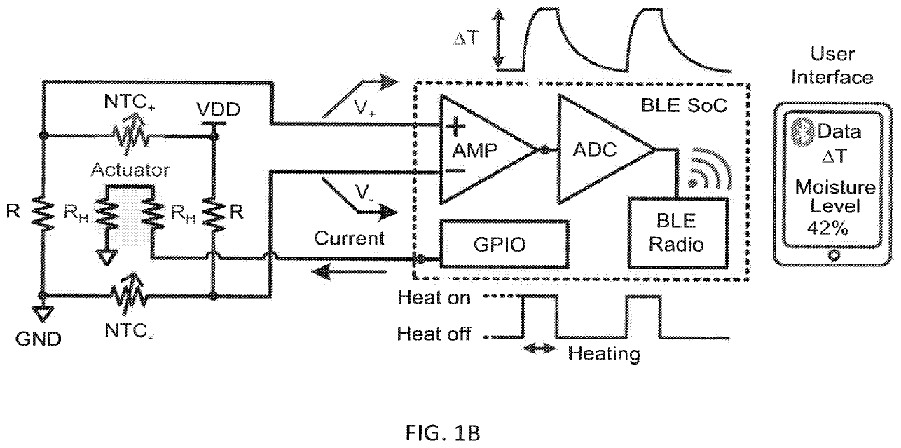

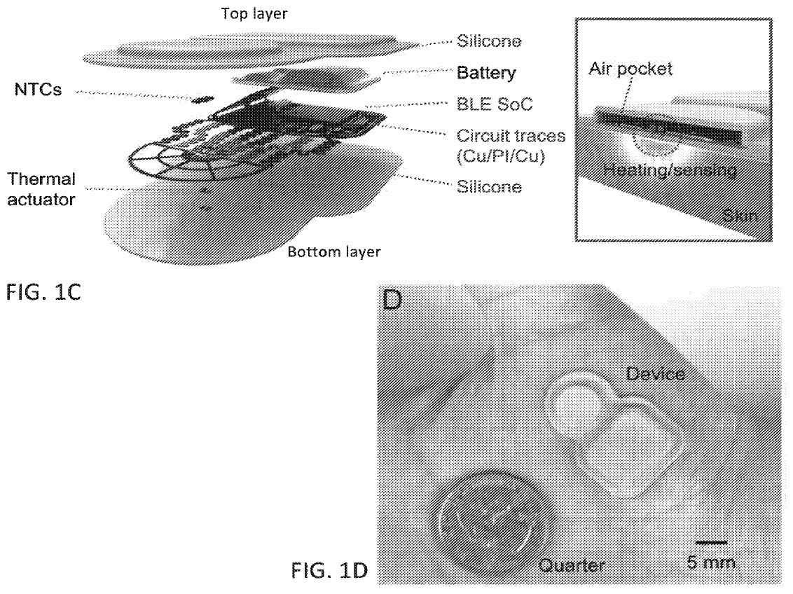

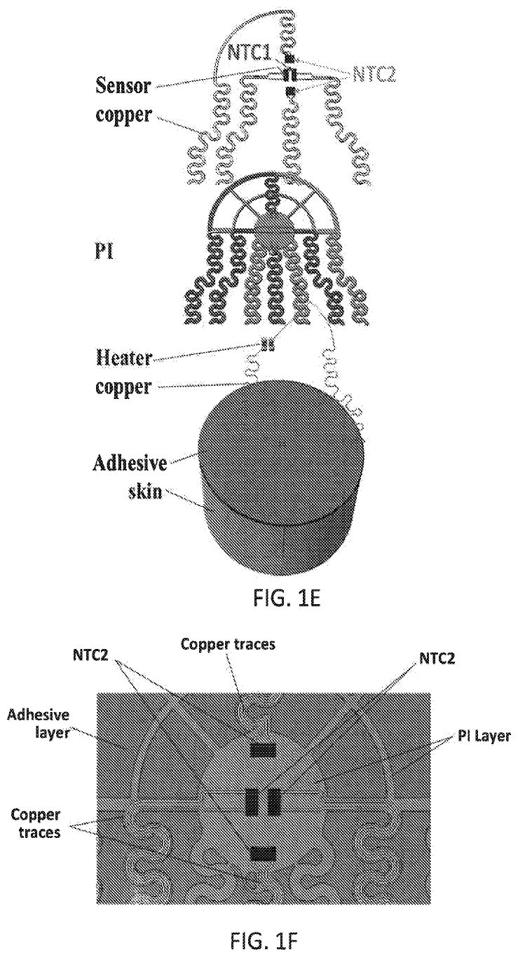

As shown in FIGS. 1A-1E, the hydration sensor comprises a sensing module, i.e., thermal actuators/sensors (TAS) module, operably disposed on a target area of interest of skin of a living subject for detecting data associated with thermal properties of the skin; and a wireless platform coupled with the sensing module for wireless data transmission between the sensing module and an external device.

The TAS module comprises a thermal actuator operably disposed on the target area of interest of the skin for heating the target area of interest thereof; and a sensing circuit for simultaneously detecting a transient temperature change (ΔT) thereof to determine thermal properties of the skin. The thermal actuator and the sensing circuit are interconnected by serpentine traces (FIG. 1E) to form a flexible structure (FIG. 1A, Inset) that facilitates soft, intimate contact to the skin with robust mechanical and thermal coupling.

In some embodiments, the thermal actuator comprises at least one resistor.

In some embodiments, the thermal actuator comprises two or more of surface-mount thin film resistors, thick film resistors, through-hole resistors, and ultrathin-film metal resistors, coupled to each other in series. As shown in FIG. 1B, the thermal actuator includes two resistors R_{H} in series.

In some embodiments, the sensing circuit comprises a temperature sensor including one or more of negative temperature coefficient thermistors, positive temperature coefficient thermistors, resistance temperature detectors (RTD), and thermocouples.

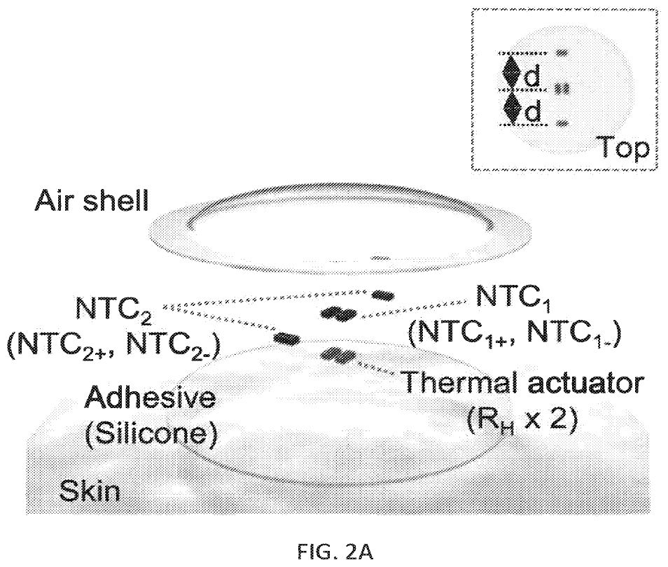

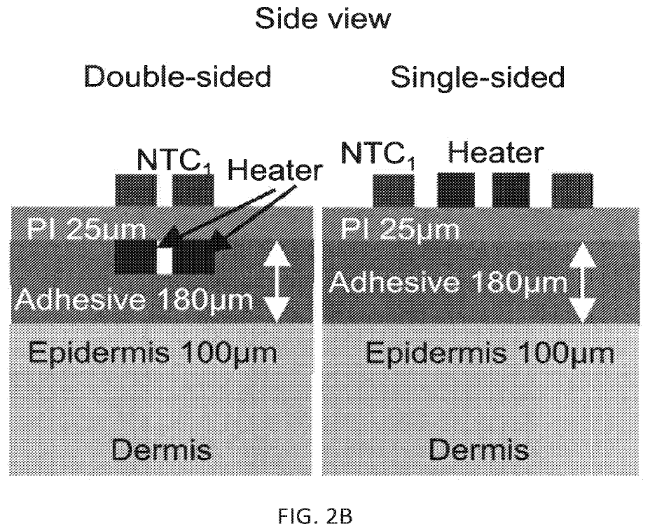

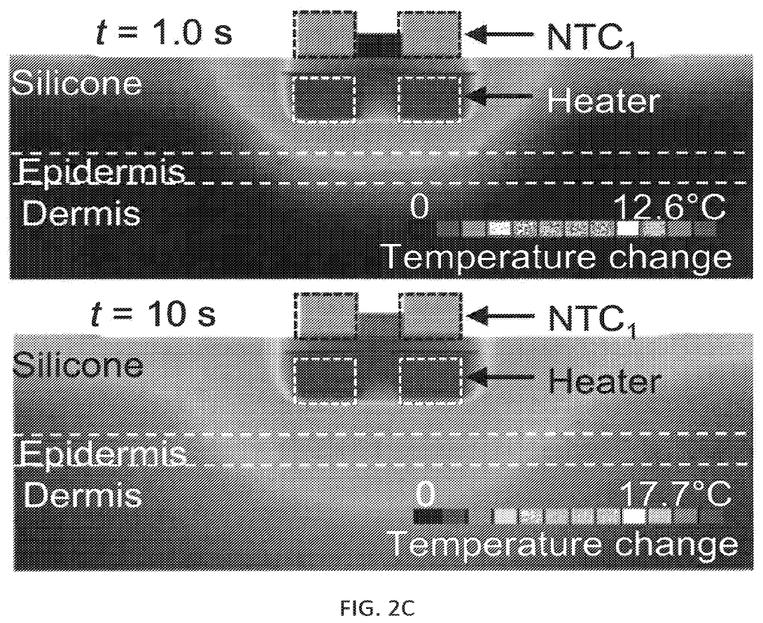

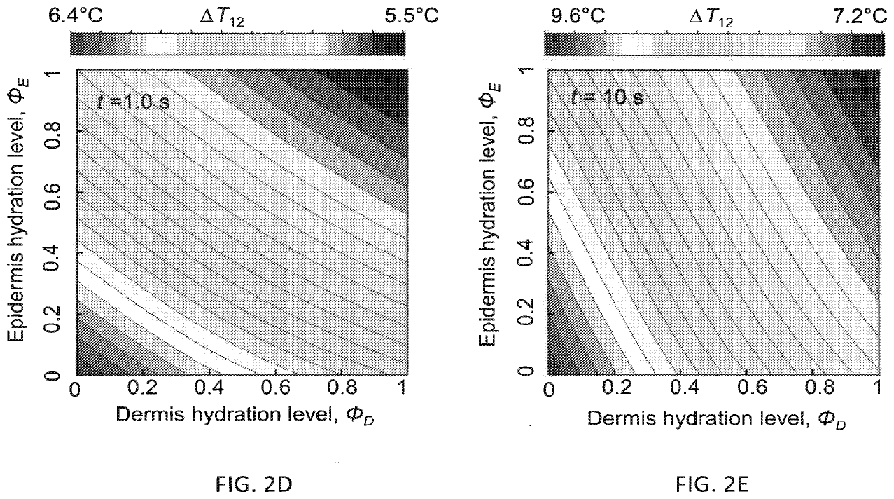

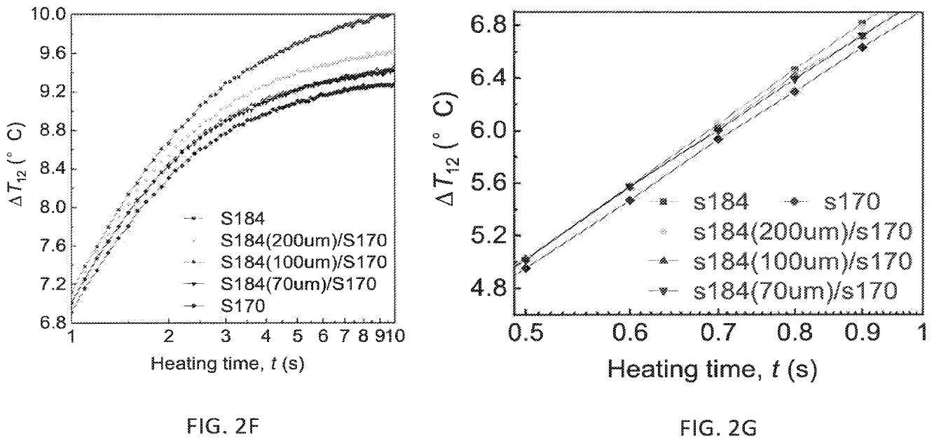

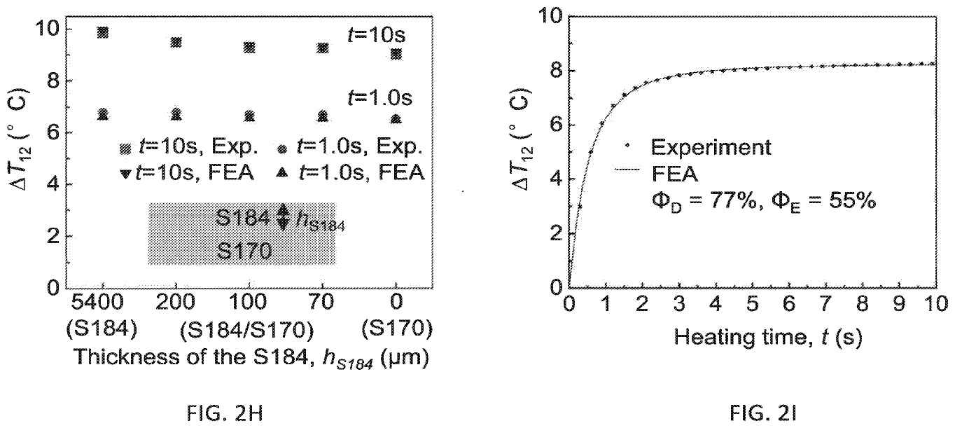

In some embodiments, the sensing circuit comprises a first pair of negative temperature coefficient thermistors NCT1 (NTC₊, NTC₋) arranged in a first Wheatstone bridge circuit, as shown in FIG. 1B. Data readings from only one sensing unit (first pair of negative temperature coefficient thermistors NCT1) may lead to fluctuating data due to the varying temperature of testing materials over time, which can be compensated by utilizing a second sensing unit. In some embodiments, the sensing circuit may also have a second pair of NTCs (NTC2, FIGS. 1E and 2A) arranged in a second Wheatstone bridge circuit serving to compensate for changes in an ambient temperature.

In one embodiment shown in FIG. 2B (Left), the first pair of NTCs (NTC1) is disposed on a layer different from the thermal actuator (heater). In this case, the two NTC1 are directly on the top of the heater. In another embodiment shown in FIG. 2B (Right), the two NTC1 are disposed on a layer same as the heater. In the case, each NTC1 has a first distance from the heater. In some embodiments, the second pair of NTCs (NTC2) is disposed on the same layer as the first pair of NTCs, and each NTC2 is spatially apart from NTC1 (FIG. 1E) and has a second distance from the heater (FIG. 2A, Inset). In some embodiments, the first and second distances are determined by the design requirement of depth sensitivity into the skin, and ranges from 10s of μm to a few mm.

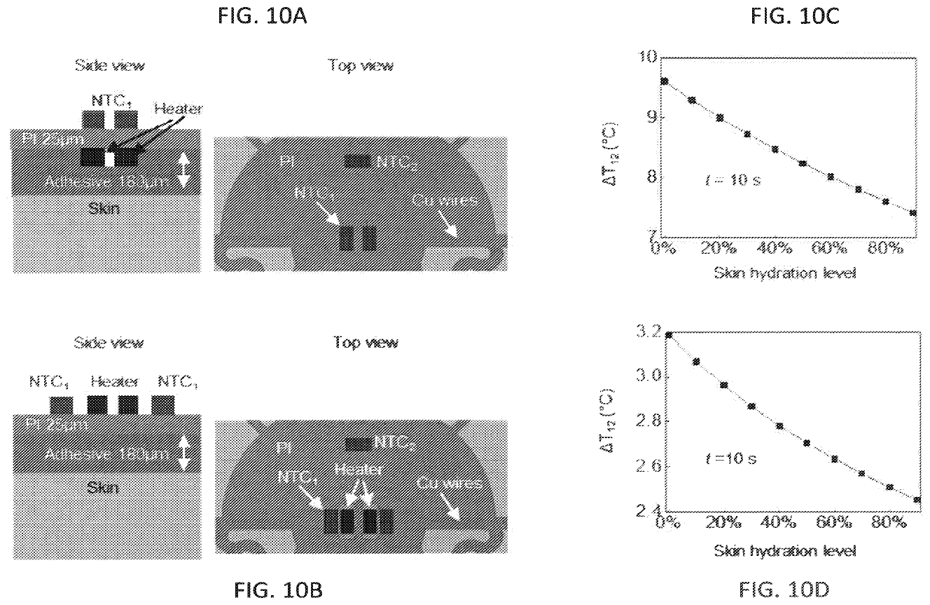

The compact, dual-sided sensor design (FIG. 2B, Left) approximately triples the sensitivity (FIGS. 10C-10D) to the hydration levels of the skin compared to a corresponding single-sided layout (FIG. 2B, Right).

In some embodiments, the wireless platform comprises at least one of Wi-Fi, BLE, and NFC communication protocols.

In one embodiment shown in FIG. 1B, the wireless platform comprises a Bluetooth low energy system on a chip (BLE SoC). In some embodiments, the BLE SoC comprises a general-purpose input/output (GPIO) electrically coupled to the thermal actuator for providing a periodic current to activate the thermal actuator; a differential amplifier (AMP) electrically coupled to the sensing circuit for amplifying a difference of bridge voltages; an analog-to-digital converter (ADC) electrically coupled to the AMP to sample/digitize output voltages of the AMP; and a BLE radio configured to wirelessly transmit output signals of the ADC to the external device for processing to determine the hydration status of the skin, and receive data from the external device to activate a GPIO pin to provide the periodic current to the thermal actuator.

Among other things, the BLE based device according to the invention provides a number of benefits including, but are not limited to, small size, automatic/remote wireless update, no need to hold the external device (e.g., phone) over the sensor, and better capability across different external devices.

In some embodiments, a digital on/off switch controlled through a custom software application including a user interface (UI) on the external device is adapted to enable BLE-connection and activation of the GPIO pin to source the periodic current into the thermal actuator.

In one exemplary embodiment, the periodic current has 6.8 mA for 10 s, and 0 mA for 50 s in a 1-min cycle. This current can generate thermal power Q=20.4 mW at the top surface of the 1 actuator and thereby delivers heat to the skin below via thermal diffusion. Transport of heat from the actuator to the NTCs depends upon the thermal properties of the skin, and thus serves as the basis for the measurement of skin hydration. Wheatstone-bridge circuits convert the resistances of the NTCs into corresponding voltages (V₊, V₋) that vary in response to changes in temperature, with an opposite polarity (ΔV₊=−ΔV₋).

In some embodiments, the custom software application in the external device transforms the voltages into corresponding temperature values based on a calibration factor. Theoretical models then convert these data into thermal transport properties of the skin, which, in turn, can be used to determine health-related parameters such as the hydration state using appropriate models.

In some embodiments, the BLE SoC may also comprise a microcontroller (μC) configured to activate the GPIO pin to source the periodic current into the thermal actuator. The μC may also be configured to process the detected data on site, and then transmit the processed data to the external device.

In some embodiments, the hydration sensor further comprises a flexible substrate in the form of a flexible printed circuit board (fPCB) with circuit traces that interconnect the thermal actuator on a skin side, the NTCs on an air side, and the BLE SoC. In some embodiments, the flexible substrate is formed of a flexible material comprising polyimide (PI) and/or polyethylene terephthalate (PET). In one example, the flexible substrate is a flexible copper-clad polyimide (Cu/PI/Cu) sheet.

In some embodiments, the hydration sensor further comprises a power module for providing power to the sensing circuit and the wireless platform. In some embodiments, the power module comprises a battery (FIG. 1C). In some embodiments, the battery is a rechargeable battery operably rechargeable with wireless recharging.

In some embodiments, the power module further comprises a wireless charging module for wirelessly charging the rechargeable battery.

In some embodiments, the power module further comprises a failure prevention element including a short-circuit protection component or a circuit to avoid battery malfunction. For example, as shown in FIG. 1C, the battery is enclosed in the encapsulation layer.

In some embodiments, the hydration sensor further comprises an encapsulating enclosure enclosing the thermal actuator, the wireless platform, the battery, and the fPCB, as shown in FIGS. 1C and 1D.

In some embodiments, the encapsulating enclosure comprises a top layer for thermal, chemical and mechanical isolation of the hydration sensor from the environment; and a bottom layer for providing a direct interface between the thermal actuator at the skin side of the fPCB and the skin.

In some embodiments, the top layer is a shell-like top encapsulation layer including small air gaps for thermally, mechanically, and chemically insulating the critical sensing components.

In some embodiments, the top layer is formed of a flexible material including silicone or silicone gel, low/high density polyethylene (LDPE/HDPE), polystyrene, Teflon®, and various other flexible polymers.

In some embodiments, the bottom layer comprises a flexible adhesive for attaching the hydration sensor to the skin.

In some embodiments, the bottom layer further comprises an ultrathin fabric of fiberglass/reinforcement material embedded in the flexible adhesive layer for enhancing the mechanical robustness of the hydration sensor. In some embodiments, the reinforcement material is flexible and has varying mesh density and thickness to lend tear resistance to the bottom layer.

In some embodiments, the flexible adhesive layer is formed of silicone or silicone gel, or double-sided skin-safe adhesives, with the ratio of silicone and silicone gel being adjusted to co-optimize mechanical integrity and tackiness of the adhesive. In one exemplary embodiment shown in FIG. 2B, the adhesive layer has a thickness of about 180 In certain embodiments, the mesh fiber/silicone bottom layer encapsulation allowing for micron to 700 micron ultra-thin bottom layer.

In some embodiments, the external device is a smartphone, a tablet, a computer, or any electronic device with data reading/processing capability, e.g., with a central processing unit (CPU), or a microcontroller unit (MCU), or an external controller.

In some embodiments, the thermal properties of the skin comprise thermal conductivity and thermal diffusivity of the skin that are related to water content of the skin, wherein the water content is a function of a skin depth.

In some embodiments, the water content is determined from the measured temperature change ΔT vs. time t.

In some embodiments, the water content and skin surface temperature are used to determine a normal state or a disease state of the skin.

In some embodiments, the water content and skin surface temperature serve as quantitative metrics of an efficacy of a treatment of a skin disease, or other health and wellness products including skin moisturizers, lotions, and/or creams.

In some embodiments, the hydration sensor is usable for monitoring the skin condition in a clinical setting and/or an at-home setting.

In some embodiments, the hydration sensor is usable for delivering treatment, monitoring the effects, modulating the treatment protocol as necessary, and/or potentially predicting for flares based on quantitative, individualized measurements on specific lesion sites.

In some embodiments, the hydration sensor is usable for monitoring water content of internal organs for various diseases where traditional monitoring techniques fail to offer continuous assessment of organ health.

In some embodiments, the hydration sensor is usable for monitoring organs during organ transport for applications in organ transplant.

In some embodiments, the hydration sensor is usable for applications to measure thermal conductivity, thermal diffusivity, heat capacity and other thermal properties of any material as a function of depth.

In some embodiments, the hydration sensor is usable for applications to measure water content of any material surface as a function of depth, including hydrogels, plants (irrigation and agriculture applications), food preservation (dried food products, grains, fruits, meats), and/or concrete (industrial applications).

In some embodiments, the hydration sensor is usable for monitoring composition of food/beverages, medicines/industrial chemicals.

In some embodiments, the hydration sensor is re-usable and removal without irritation to the skin or damage to the hydration sensor.

In some embodiments, the hydration sensor is compatible with alcohol-based cleaning wipes allowing for re-use across different users, without any damage to the hydration sensor or loss in efficacy of the hydration sensor adhesive.

In some embodiments, the hydration sensor is sterilizable using alcohol, autoclave steam sterilization, and gas phase sterilization.

In another aspect, the invention relates to a method of fabricating a hydration sensor. In some embodiments, the method includes forming a flexible printed circuit board (fPCB) that interconnects electronics of the hydration sensor; and forming an encapsulating enclosure enclosing the sensing module, the wireless platform and the fPCB. The encapsulating enclosure comprises a top layer and a bottom layer.

In some embodiments, the fPCB is formed of a flexible material comprising polyimide (PI), polyethylene terephthalate (PET), or any one of them in combination with stiff PCB material including FR-4.

In some embodiments, the bottom layer comprises a layered structure of a first flexible layer, a second flexible layer, and a fabric of fiberglass/a reinforcement material embedded between the first flexible layer and the second flexible layer.

In some embodiments, each of the first flexible layer and the second flexible layer is formed of silicone or silicone gel, or double-sided skin-safe adhesives, with the ratio of the silicone and silicone gel being adjusted to co-optimize mechanical integrity and tackiness of the adhesive.

In some embodiments, the reinforcement material is flexible and has varying mesh density and thickness to lend tear resistance to the bottom layer.

In some embodiments, the bottom layer adheres to the f-PCB through use of silicone bonding material, epoxy, glue, or commercial adhesive.

In some embodiments, the top shell layer is formed of silicone or silicone gel, low/high density polyethylene (LDPE/HDPE), polystyrene, Teflon®, and various other flexible polymers.

In some embodiments, the electronics comprises a sensing module for detecting data associated with thermal properties of the skin; and a wireless platform coupled with the sensing module for wireless data transmission between the sensing module and an external device.

In some embodiments, the sensing module comprises a thermal actuator for heating a target area of interest of the skin; and a sensing circuit for simultaneously detecting a transient temperature change (ΔT) thereof to determine thermal properties of the skin.

In some embodiments, the wireless platform comprises at least one of Wi-Fi, BLE, and NFC communication protocols.

In some embodiments, the wireless platform comprises a BLE SoC.

In yet another aspect, the invention relates to a method of monitoring and/or diagnosing a condition of a skin. In some embodiments, the method comprises attaching a hydration sensor onto a target area of interest on the skin, wherein the hydration sensor comprises a thermal actuator, a sensing circuit, and a wireless platform for two-way data communication with an external device; heating the target area of interest of the skin by the thermal actuator, simultaneously detecting data associated with thermal properties of the skin by the sensing circuit, and wirelessly transmitting the detected data, by the wireless platform, to the external device to determiner a transient temperature change (ΔT) thereof; obtaining water content of the target area of interest of the skin from the temperature change (ΔT); and determining a condition of the skin at the target area of interest based on the obtained water content.

In some embodiments, the water content comprises water content Φ_{E} of the epidermis and water content Φ_{D} of the dermis.

In some embodiments, the step of obtaining the water content comprises separately determination of Φ_{E} and Φ_{D} from the temperature change ΔT.

In some embodiments, the wireless platform transmits data through a wireless communication protocol including Near Field Communication (NFC), Wi-fi/Internet, Bluetooth/Bluetooth low energy (BLE), or GSM/Cellular Communication.

In some embodiments, said heating the target area of interest of the skin is formed by providing a periodic current to the thermal actuator.

In some embodiments, activation of the periodic current is controlled by a digital on/off switch through a custom application on the external device.

In some embodiments, said determining the condition of the skin at the target area of interest comprises comparing the obtained water content to a standard water content at the target area of interest so as to determine a normal state or a disease state of the skin.

In some embodiments, said determining the condition of the skin at the target area of interest comprises diagnosing a skin disease at the target area of interest based on wherein the obtained water content thereof.

In some embodiments, said determining the condition of the skin at the target area of interest comprises evaluating an efficacy of a treatment of the skin disease.

In some embodiments, said obtaining water content of the target area of interest of the skin, and said determining a condition of the skin are performed in the external device.

In some embodiments, the method further comprises displaying the condition of the skin at the target area of interest in the external device.

In some embodiments, the method further comprises forwarding the condition of the skin at the target area of interest, or sending an alert, to a professional, a caregiver and/or a service provider. For example, important biological tissue parameters may be obtained, even for a user outside of a controlled medical setting. Those parameters may be communicated at a distance for evaluation in real-time, or at a later time, such as by the user or a third party, such as a medical caregiver, friend or family member. The devices and methods are also compatible with a more active intervention, ranging from a warning provided to the user to an automated response, such as application of a hydrating compound, sun block compound, or any other response depending on the application of interest.

In some embodiments, the method further comprises one or more steps of delivering treatment, monitoring the effects, modulating the treatment protocol as necessary, and/or potentially predicting for flares based on quantitative, individualized measurements on specific lesion sites.

In some embodiments, measurement conductions arc optimized to obtain accurate and reproducible results. Accordingly, the method can be performed under one or more of the optimized measurement conditions: (1) the measurement is performed rapidly, to minimize effects of occlusion of natural processes of water vapor release from the skin due to the presence of the hydration sensor; (2) very light or zero applied pressure is used during the measurement, to minimize perturbations to the skin; (3) the adhesive is patterned such that it is present only across regions of the hydration sensor device adjacent to the sensor itself, to avoid exfoliation of the skin at the measurement site during peel back, for improved repeatability; (4) the temperature of the hydration sensor is comparable to that of the skin; and (5) skin itself is allowed to acclimate to the surrounding environment prior to the measurement.

Wireless electronics for monitoring of skin hydration in a quantitative fashion have broad relevance to the understanding of dermatological health and skin structure in both clinical and home settings. According to the invention, the miniaturized, long-range automated system that adheres gently to the skin to yield quantitative recordings of skin water content for both epidermis and dermis supports capabilities in characterizing skin barrier, assessing severity of skin diseases, and evaluating cosmetic and medication efficacy, with high levels of repeatability and insensitivity to ambient. Benchtop and pilot studies on patients with skin diseases highlight key features of these devices and their potential for broad utility in clinical research and in home settings to guide the management of disorders of the skin. These and other aspects of the present invention are further described in the following section. Without intending to limit the scope of the invention, further exemplary implementations of the present invention according to the embodiments of the present invention are given below. Note that titles or subtitles may be used in the examples for the convenience of a reader, which in no way should limit the scope of the invention. Moreover, certain theories are proposed and disclosed herein; however, in no way should they, whether they are right or wrong, limit the scope of the invention so long as the invention is practiced according to the invention without regard for any particular theory or scheme of action.

### Example

### Wireless, Soft Electronics for Rapid, Multisensor Measurements of Hydration Levels in Healthy and Diseased Skin

In this exemplary example, wireless systems for performing hydration level measurements routinely and reliably in healthy and diseased skin are disclosed. Computational methods applied to the resulting data define the hydration levels using bilayer models for the skin, with clinical-grade levels of accuracy. Relative to other technologies, important advances of the wireless systems include, but are not limited to, 1) long-range wireless capabilities and high sampling rates with Bluetooth interfaces to the phone; 2) compact, dual-sided multi-sensor designs with enhanced sensitivity to the hydration levels of the skin; 3) multiple, redundant measurement modalities with minimized susceptibility to parasitic environmental, physiological, and user-related factors; and 4) full-waveform data analysis techniques with ability to determine hydration levels of both the epidermis and dermis, and with additional sensitivity to the SC. Numerical modeling results and benchtop characterization tests under various practical conditions define the key physical effects and guide the selection of optimized designs and modes of operation. Validation studies involve porcine skin with known levels of hydration and human subjects with benchmarks against clinical devices. Here and in other practical scenarios, the soft mechanical properties and compliant construction of the sensor enable intimate coupling to the skin without applied force and hold, to avoid angle or pressure-related sources of variability that degrade the accuracy and repeatability of conventional devices. These same features in form factor and performance allow for routine measurements at nearly any location on the body and on subjects of any age.

This collective set of attributes forms the foundations for devices that allow rapid, accurate assessments of skin hydration and skin barrier function with almost zero user burden. Simple interfaces that leverage smartphone technology suggest potential for frequent use in home settings, as preemptive management of skin disease prior to flares for conditions such as AD or XC. Pilot scale clinical studies illustrate these and other capabilities in tracking improvements in skin hydration associated with application of topical moisturizers onto patients with a range of inflammatory skin conditions. Overall, this system has the potential to improve the quality of care for patients by providing objective and accurate measurements of skin barrier function.

### Materials and Methods

Fabrication of the Electronics: Prototype devices, as shown in FIGS. 1A, 1C and 1D, used flexible copper-clad polyimide substrates (AP8535R; Pyralux) processed by laser ablation (Protolaser U4; LPKF), resulting in flexible printed circuit boards (fPCBs) to interconnect surface-mount components, including a BLE SoC (nRF52832; Nordic Semiconductor), resistors (RMCF0201FT; Stackpole Electronics), and temperature sensors (NTC; NCP03XH; Murata). Outcomes of studies of the prototype fPCBs served as the basis for designs provided to an ISO-9001 compliant vendor (PCBWay) for final designs. Soldering wire (MM01019; Multicore) and soldering paste (SMDLTLFP10T5; Chip Quik) bonded the BLE SoC to the fPCB by heating at 400 C, and other various surface-mount components by heating at 190 C.

Software Development Environment: A BLE mesh kit board (nRF52 DK; Nordic Semiconductor) facilitated development of software for the BLE SoCs. A PC connected to the nRF52 DK with a USB cable for power enabled programming of the on-board BLE SoC. A source-code editor (Visual Studio Code; Microsoft) supported authoring, modifying, compiling, deploying, and debugging software of BLE SoC. A power profiler kit board (NRF6707; Nordic Semiconductor) interfaced with nRF52 DK provided real-time measurements of current consumption of the embedded applications. Android's official integrated development environment (IDE) (Android Studio; Google) provided tools to develop and build the custom Android application (user interface) on smartphones.

Design of the Encapsulating Enclosure: A triple-layered structure of silicone (Ecoflex 00-30; Smooth-On)/silicone gel (Ecoflex gel; Smooth-On), fiberglass fabric (optional, not shown explicitly in FIG. 1C), and a different formulation of silicone (Silbione RTV 4420; Elkem Silicones) (80 μm/20 μm/80-μm thickness) served as the bottom encapsulation layer of the device. The bottom adhesive silicone/silicone gel layer provided a direct interface between the heater at the bottom side of the fPCB and the skin, as formed using a three-step process: 1) spin-coating the silicone/silicone gel layer with 2,500 rpm for 30 s on a glass slide and curing on a hot plate at 85 C for 10 min, 2) gently placing a fiberglass fabric on top of the silicone/silicone gel layer, and 3) spin-coating the following silicone layer with 1,500 rpm for 30 s, placing the device on the uncured silicone layer with the heater side facing down, and curing on a hot plate at 85 C for 10 min to achieve adhesion between the fPCB and the silicone layer. Curing the silicone (Silbione RTV 4420; Elkem Silicones) inside a custom-made aluminum mold on a hot plate at 85 C for 20 min formed the top shell of the device (˜4-mm height). Curing the top shell and bottom layer together on a hot plate at 85 C for 20 min with a small amount of silicone (Silbione RTV 4420; Elkem Silicones) as an adhesive sealed the entire system. Cutting the structure using a die cutter completed the fabrication process. Proper cleaning (contaminants/debris removal) using alcohol swipes (Sterile Alcohol Prep Pads; Dynarex) restores the adhesion due to van der Waals forces. Additional adhesives (3M 1524, 3M Tegaderm, etc.) can be used to improve adherence to the skin, as necessary.

Adhesive Stripping Measurement: Repeated application and removal adhesive disks (D-Squame; CuDerm; 14-mm diameter, ˜100-μm thickness) several times on the same area of skin gradually removed the SC. Replacement of each disk occurred after five cycles. Measurements after 0, 10, 20, and 35 cycles involved two commercial devices (MoistureMeterSC and MoistureMeterD; Delfin Technologies) and the BLE device.

Porcine Skin Water-Loss Measurement: DPBS solution (Gibco Dulbecco's phosphate-buffered saline; 14190-136; Life Technologies) defrosted a piece of porcine skin (˜25-mm thickness; 200×100 mm) at room temperature for 12 h. A commercial dehydrator (Sedona Combo Rawfood Dehydrator SD-P9150; Tribest) controlled the hydration level of the porcine skin at 33 C for 10 min for each measurement. Measured weights of the porcine skin determined with a balance (Ohaus Ax622 Adventurer Precision Balance; Ohaus) enabled a calculation of water loss.

Human Subject Evaluations: The objective was to validate a BLE-based skin hydration monitor as a capable detector of differences in thermal conductivity between dry/hydrated skin and tissue affected and unaffected by skin diseases such as atopic dermatitis. The sensor represents low to minimal risk to the patient, with no electrical component touching the skin. More than 10 healthy control adults/children and 3 patients with mild, moderate, or severe atopic dermatitis were engaged in a dermatology clinic and measured with the sensor by placing it on the skin at discrete locations of the body. The baseline reference for determining TEWL of skin was also obtained using commercially available devices based on capacitance measurements of a dielectric medium in skin.

Body locations selected for studies included the forehead, left/right forearm, and left/right lower leg. Conventional devices with different probing depths provided baseline references for skin hydration in triplicate on each body location prior to the BLE measurements. A 5-min, continuous measurement using the BLE device were then performed, without the need for a waiting period for the sensor to reach thermal equilibrium with the skin. During the BLE measurements, the subjects were allowed to move freely without any constraint on activities. The tests were performed indoor under an air-conditioned environment.

Moisturizer studies on two patients with AD. The experimental protocol involves four steps: 1) perform three measurements on disease-affected and unaffected skin, 2) apply a moisturizer (Extremely Dry Skin Rescue Lotion; Vaseline) and wait for 15 min, 3) wipe away excess moisturizer from the surface of the skin, and 4) repeat three measurements at each location.

Moisturizer studies on three healthy adults. A thin standardized layer (˜1 to 2 g/cm²) of a commercially available, fragrance-free moisturizer was applied to each location. Repeat measurements were performed at 1 min and 15 min after application of the moisturizer.

Patients (Ann and Robert H. Lurie Children's Hospital of Chicago, Chicago, Ill.) and healthy/normal subjects (Northwestern University, Evanston, Ill.) recruited were voluntary and provided full informed consent. This study was approved by the Northwestern University institutional review board (IRB) (IRB study STU00209010). Single-use alcohol wipes (Sterile Alcohol Prep Pads; Dynarex) provided sterilization of the BLE and commercial devices.

### Results and Discussion

System Configurations: The device (FIG. 1A, shown here without the battery) is a small, wireless platform designed for noninvasive measurements of the temperature and the thermal transport properties of the skin. The width, length, height, and weight of this example, excluding a battery, are 14.6 mm, 25.6 mm, 1.2 mm, and 193.0 mg, respectively. The system includes a thermal actuator and multisensor (TAS) module interconnected by serpentine traces to form a flexible structure that facilitates soft, intimate contact to the skin with robust mechanical and thermal coupling (Inset). FIG. 1B presents circuit and block diagrams that highlight the Bluetooth Low Energy (BLE) system on a chip (SoC) for control and wireless data communication to a user interface (UI) (typically on a portable device such as a smartphone). The TAS module includes a thermal actuator (Joule heating through 221 Ω×2 resistors; R_{H}×2) and Wheatstone bridge circuits with a pair of negative temperature coefficient thermistors (NTC₊, NTC₋) and a known resistor (R) on each bridge for primary measurement purposes. Another pair of NTCs and bridge circuit serves to compensate for changes in the ambient temperature. A digital on/off switch controlled through the UI enables BLE-connection and activation of a general-purpose input/output (GPIO) pin to source a periodic current (6.8 mA for s, and 0 mA for 50 s in a 1-min cycle) into the thermal actuator. This current generates thermal power (Q=20.4 mW) at the top surface of the structure and thereby delivers heat to the skin below via thermal diffusion. Transport of heat from the actuator to the NTCs depends upon the thermal properties of the skin, and thus serves as the basis for the measurement of skin hydration. Wheatstone-bridge circuits convert the resistances of the NTCs into corresponding voltages (V₊, V₋) that vary in response to changes in temperature, with an opposite polarity (ΔV₊=−ΔV₋). This configuration supports enhanced sensitivity compared to schemes used in conventional implementations of TPS methods. A differential amplifier (AMP) in the BLE SoC further amplifies the voltage differences while eliminating common-mode noise to increase the signal-to-noise ratio (SNR). The subsequent analog-to-digital converter (ADC) samples the voltage, for transmission to the UI via BLE radio communication protocols. A software application transforms the voltages into corresponding temperature values based on a calibration factor. Theoretical models then convert these data into thermal transport properties of the skin, which, in turn, can be used to determine health-related parameters such as the hydration state using appropriate models.

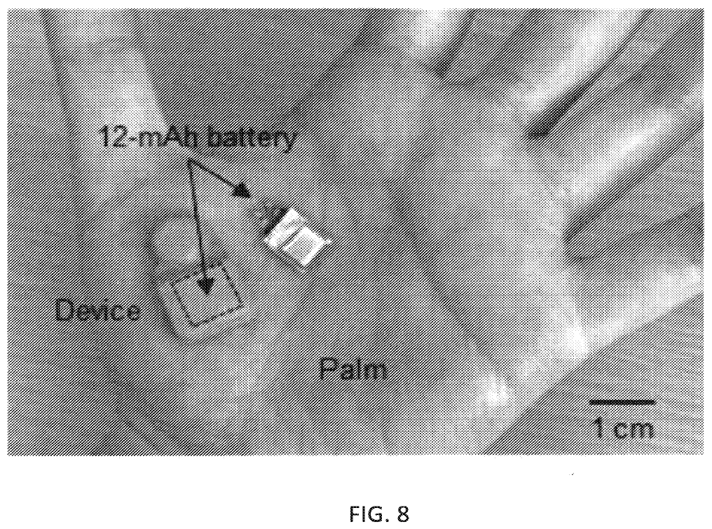

The exploded view schematic illustration in FIG. 1C highlights the constituent layers and components of the system: a shell structure formed in a biocompatible silicone material for packaging and thermal insulation; a Li-polymer battery (12 mAh); and a flexible copper-clad polyimide substrate (AP8535R; Pyr-alux) processed by laser ablation (Protolaser U4; LPKF) to define circuit traces that interconnect the thermal actuator (skin side), NTCs (air side), and the BLE SoC. The shell (Inset) creates an air pocket around the TAS module to optimize the flow of heat to the skin-facing side of the device and to thermally isolate this region from the ambient. The miniaturized dimensions of the TAS module (width and length of 0.9 and 2.6 mm, respectively) facilitate proper alignment and compliance with the skin, as needed for accurate measurements. A picture of an encapsulated device adhered to the thenar eminence is in FIG. 1D. Assuming that the system performs temperature measurements at a 200-Hz sampling rate, transmits an averaged value every 0.1 s (10 Hz) to the UI, and measures the hydration state over 1 min (actuator off for 50 s, on for 10 s) per day, a 12-mAh battery (FIG. 8) supports an expected lifetime of nearly 10 days.

In this exemplary embodiment, the system does not show capabilities in wireless recharging, such functionality can be easily included. For example, in certain embodiments, the system may include a rechargeable battery, which can be wirelessly recharged through a wireless battery charging module.

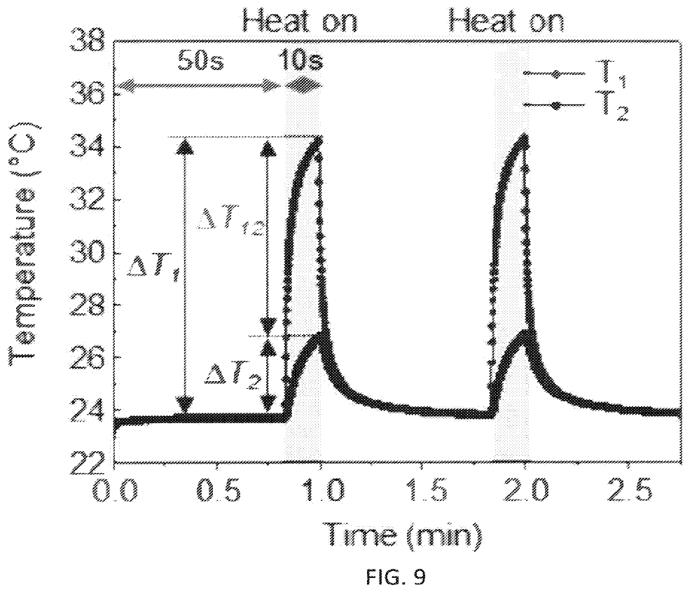

Thermal Transport Physics and Applications to Measurements of Skin Hydration: Standard modules for TPS measurements capture the time-dependent difference in temperature (ΔT) for cases when the thermal actuator is off and on (T_{off} and Tₒₙ, respectively). The simplest approach to analysis uses a value ΔT=Tₒₙ−T_{off} at a single time point, often in a quasi-steady-state regime where the rate of change of temperature with time is relatively small. This parameter then determines an effective thermal transport characteristic, using appropriate models and calibration procedures. Measurement and analysis of the full-time dependence, starting immediately after the actuator is turned on and continuing to the quasi-steady-state regime, can yield substantial information on thermal transport, as described subsequently. In all cases, changes in skin temperature or variable environmental conditions (air currents, ambient temperature fluctuations) that may occur between or during the measurements of T_{off} and Tₒₙ within a given measurement cycle can affect the value of ΔT, thereby degrading the accuracy and precision of the system. A key feature of the TAS module in some embodiments is that it includes two pairs of NTCs (NTC₁ and NTC₂), as shown in FIG. 2A. The primary measurement exploits the difference of the values of ΔT measured from NTC₁ and NTC₂ (ΔT₁ and ΔT₂, respectively; FIG. 9), or ΔT₁₂=ΔT₁−ΔT₂=(T_{on,1}−T_{off,1})−(T_{on,2}−T_{off,2}). Here, NTC₂ captures the temperature at a location distant from the thermal actuator, to eliminate the effects of uncontrolled temperature fluctuations, as demonstrated under various conditions in subsequent sections.

An exploded-view illustration of the TAS module (FIG. 2A) highlights the constituent layers and components: adhesive (180 μm thickness), thermal actuator (two resistors in series; R_{H}×2), NTC₁ (NTC₁₊ and NTC₁₋), NTC₂ (NTC₂₊ and NTC₂₋), and silicone encapsulation with air shell. The Inset shows a top view of the assembled module. NTC₁ and NTC₂ are directly above and 1.15 mm away from the center of the thermal actuator, respectively. The widths (w) and lengths (l) of both R_{H} and the NTCs are 0.3 and 0.6 mm, respectively. The compact, dual-sided sensor design (FIG. 2B, Left) approximately triples the sensitivity (FIGS. 10A-10D) to the hydration levels of the skin compared to a corresponding single-sided layout (FIG. 2B, Right). FIG. 2C shows the temperature distribution obtained from the finite element analysis (as described in Macroscale Modeling by Finite Element Analysis (FEA) below) induced by operation of the thermal actuator (Q=20.4 mW) for skin with volumetric composition of 50% of water (kₛₖᵢₙ=0.35 W·m⁻¹·K⁻¹ and αₛₖᵢₙ=0.125 mm²·s⁻¹) at short (t=1.0 s; top) and long (t=10 s; bottom) times following initiation of heating. For a typical epidermal thickness (h=100 μm), at short times (e.g., t=1.0 s), thermal transport substantially occurs only through the epidermis. For long times (e.g., t=10 s), heat passes through the epidermis and significantly into the dermis. Modeling the skin as a bilayer of epidermis (E) and dermis (D) enables extraction of the approximate, averaged hydration level of each layer individually from the ΔT₁₂ measurement. At the macroscale, ΔT₁₂ (at time t=0 to ˜10 s) can be derived from the thermal properties (thermal conductivity, k; and diffusivity, α) of the epidermis (E) and dermis (D), i.e., k_{E}, α_{E}, k_{D}, and α_{D}, with a quantitative correlation established via FEA modeling. A microscale model of hydrated skin (as described in Micromechanics Model for the Thermal Properties of Hydrated Skin below) defines relationships between k and a, such that the thermal transport problem can be solved with only two parameters to be determined, i.e., the hydration levels Φ_{E} and Φ_{D} of the epidermis and dermis, respectively. At short times (e.g., t=1.0 s), ΔT₁₂ is more sensitive to Φ_{E} than Φ_{D}, as shown in FIG. 2D. Conversely, at long times (e.g., t=10 s), ΔT₁₂ is more sensitive to Φ_{D} than Φ_{E}, as shown in FIG. 2E. Analysis at these two time regimes, or throughout the entire measurement period, can yield both Φ_{D} and Φ_{E}.

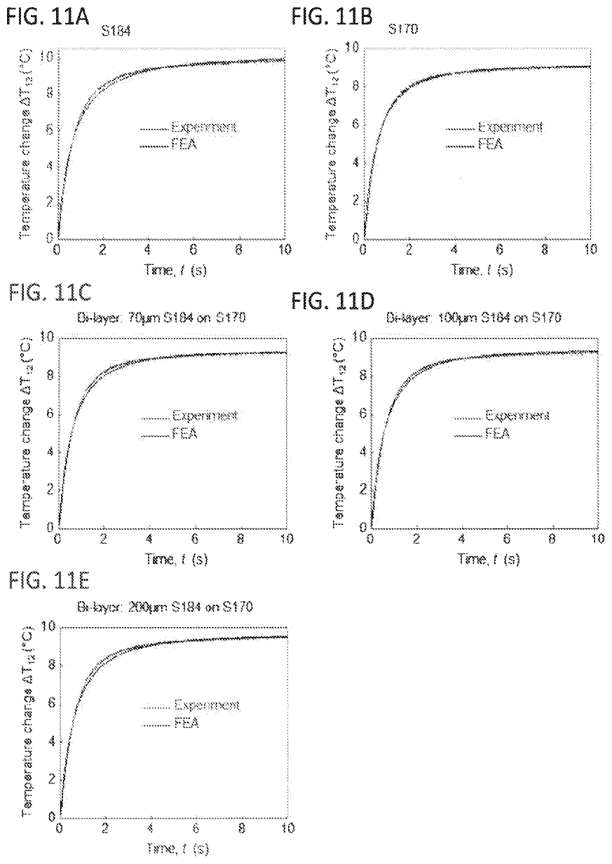

Test structures built with formulations of poly(dimethylsiloxane) (PDMS) that have thermal transport properties similar to those of dehydrated (S184) and hydrated (S170) skin illustrate the key effects. FIGS. 2F-2G show wireless measurements of ΔT₁₂ at long (FIG. 2F; t=1 to ˜10 s) and short (FIG. 2G; t=0.5 to ˜1 s) times, respectively, for samples that include a thick layer of S184 (red) and S170 (blue), and a thin layer of S184 (70 μm, black; 100 μm, green; 200 μm, yellow) on top of the S170. At long times (e.g., t=10 s), heat passes through the top layer (˜100-μm thickness) and substantially penetrates into the bottom substrate. This process leads to similar values of ΔT₁₂ for the S184/S170 structures and those of S170. On the other hand, at short times (e.g., t=0.5 to ˜1 s; FIG. 2G), the heat generated from the device remains confined to the top layer (˜100-μm thickness), where ΔT₁₂ from the S184/S170 structures are similar to those of S184. FIGS. 11A-11E highlight the FEA results and experimental data for samples described above. The FEA results agree well with the experimental values (Exp.) with SDs less than 3.5%, as shown in FIG. 2H. These effects support a mode of operation in which analysis of data across different time intervals allow for separate measurements of the hydration of the epidermis and the dermis. FIG. 2I represents results for ΔT₁₂ from the forearm throughout the entire measurement period (t=0 to ˜10 s). The extracted values of Φ_{D} and Φ_{E}, are consistent with the expected water content at different skin depths. Similar considerations applied to further reduced time intervals allow for separate measurements of the SC and the epidermis. Unless stated otherwise, the studies described in the following use, for simplicity, a single measurement at 10 s (i.e., ΔT₁₂ at 10 s) with a single-layer skin model. The reported values of hydration, referred to as Φ (Φ=0 for dehydrated skin, and Φ=1 for water), correspond to a weighted average of Φ_{D} and Φ_{E}. The measurement characteristics also depend on the layouts and sizes of the thermal actuator and sensor components, as described in detail in a Simplified Analytical Model below.

Macroscale Modeling by Finite Element Analysis (FEA): At the macroscale, FEA establishes a relationship between ΔT₁₂ and the thermal conductivity and thermal diffusivity of the epidermis and dermis (k_{E}, α_{E}, k_{D} and α_{D}) based on the transient heat transfer analysis using the software ABAQUS. A schematic illustration of the FEA model is given in FIG. 10A. A refined mesh (˜1 million elements) with mesh size much smaller than the finest feature size of the device (18 μm, copper thickness) and a refined time increment that limits the maximum temperature change to below 0.5° C. in each increment ensure the simulation convergence and accuracy. The literature values of the material parameters are k_{copper}=377 W/(m-K), α_{copper}=109 mm²/s, kPI=0.55 W/(m-K), αPI=0.32 mm²/s, k_{Ecoflex}=0.21 W/(m-K), α_{Ecoflex}=0.11 mm²/s. The thermal conductivity of polyimide (PI) is determined as kPI=0.55 W·m⁻¹·K⁻¹ from the measurements on a material with known thermal properties (S170, k_{S170}=0.40 W·m⁻¹·K⁻¹, α_{S170}=0.14 mm²·s⁻¹). For validation, a different material (S184) with known thermal properties (k_{S184=0.20} W·m⁻¹·K⁻¹, α_{S184}=0.11 mm²·s⁻¹) and the bi-layer material of thin S184 (70˜200 μm thickness) on thick S170 are tested, and the FEA results agree well with experiments without any additional fitting (FIGS. 11A-11E).

Micromechanics Model for the Thermal Properties of Hydrated Skin: A micromechanics model establishes a relationship between the thermal properties of hydrated skin and its hydration level 0 (volumetric water content). The hydrated skin is modeled as a composite of dry skin (thermal conductivity k_{dry}=0.2 W·m⁻¹·K⁻¹, thermal diffusivity α_{dry}=0.15 mm²·s⁻¹) and water (kW=0.6 W·m⁻¹·K⁻¹, αW=0.14 mm²·s⁻¹) which gives the thermal conductivity kₛₖᵢₙ and thermal diffusivity αₛₖᵢₙ of the hydrated skin as

$$\frac{{k}_{skin}}{{k}_{dry}} = \frac{( p + 2 ) + 2 ( p - 1 ) Φ}{( p + 2 ) - ( p - 1 ) Φ} , p = \frac{{k}_{W}}{{k}_{dry}} ,$$

$$\frac{{α}_{skin}}{{α}_{dry}} = \frac{{α}_{W} {k}_{skin}}{( 1 - Φ ) {α}_{W} {k}_{dry} + Φ {α}_{dry} {k}_{W}} ,$$

respectively.

For the bi-layer model of the epidermis and dermis layers for the skin, the above micromechanics model applies to each layer, with the subscript ‘skin’ replaced by ‘E’ and ‘D’ for epidermis and dermis, respectively.

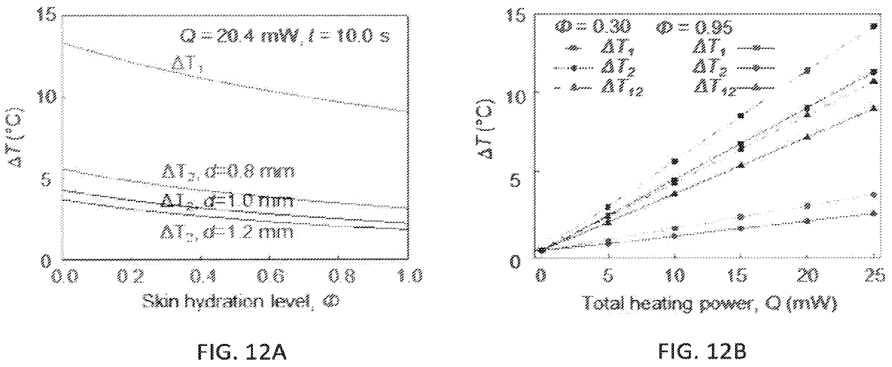

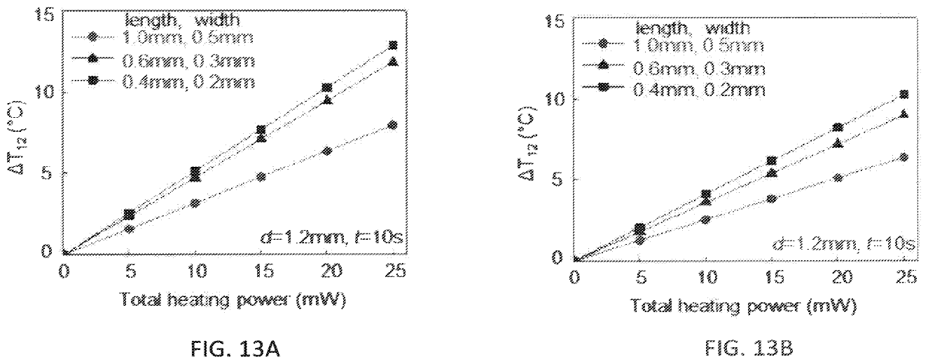

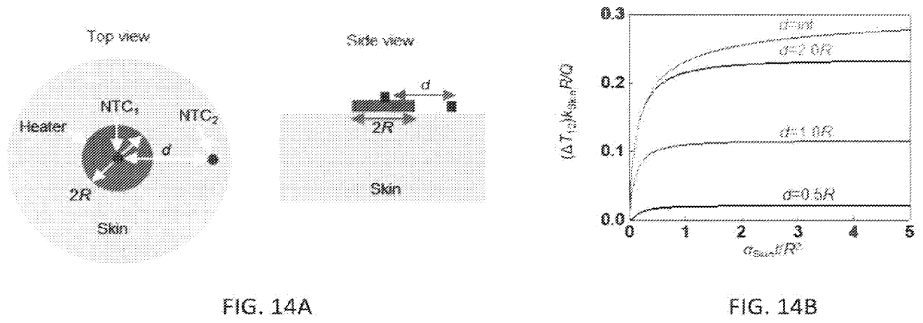

A Simplified Analytical Model: A simplified model for the relationship between the NTC₁-to-NTC₂ spacing and their temperature difference is useful. The data in FIGS. 12A-12B correspond to FEA results for ΔT (Q=20.4 mW, t=10 s) as a function of cb with different distances (d) between NTC₁ and NTC₂, and ΔT as a function of Q (t=10 s, d=1.2 mm, Φ=0.3). The value of ΔT₁₂ increases as d and Q increase, and as b decreases. The effect of actuator size (width and length of R_{H}) on ΔT₁₂ is in FIGS. 13A-13B. As shown in FIG. 14A, a disk-shaped heater (radius R and heating power Q) and two infinitesimal sensors rest on a semi-infinite, homogenous substrate with the properties of skin (thermal conductivity kₛₖᵢₙ and thermal diffusivity αₛₖᵢₙ). The heater and sensors have negligible thicknesses. The position of NTC₁ is directly above the heater (r=0 in the polar coordinate system) and NTC₂ is at a distance d from NTC₁. The temperature changes in NTC₁ and NTC₂ are

$$Δ {T}_{1} = \frac{Q}{π R {k}_{skin}} {\int }_{0}^{∞} [ {J}_{1} ( x ) erfc ( \sqrt[- x]{\frac{t {α}_{skin}}{{R}^{2}}} ) ] \frac{dx}{x} ,$$

$$Δ {T}_{2} = \frac{Q}{π R {k}_{skin}} {\int }_{0}^{∞} [ {J}_{0} ( \frac{xd}{R} ) {J}_{1} ( x ) erfc ( \sqrt[- x]{\frac{t {α}_{skin}}{{R}^{2}}} ) ] \frac{dx}{x} ,$$

respectively, where J₀(x) and J₁(x) are Bessel functions of the first kind with zero- and first-orders, respectively, and erfc(x) is the complementary error function. Therefore, the temperature difference between the two sensors can be expressed in the following dimensionless form

$$\frac{( Δ {T}_{1} - Δ {T}_{2} ) {Rk}_{skin}}{Q} = \frac{1}{π} f ( \frac{t {α}_{skin}}{{R}^{2}} , \frac{d}{R} ) .$$

The function f is plotted in FIG. 14B. The measurement sensitivity increases with

$$\frac{t {α}_{s k i n}}{{R}^{2}} or \frac{d}{R} .$$

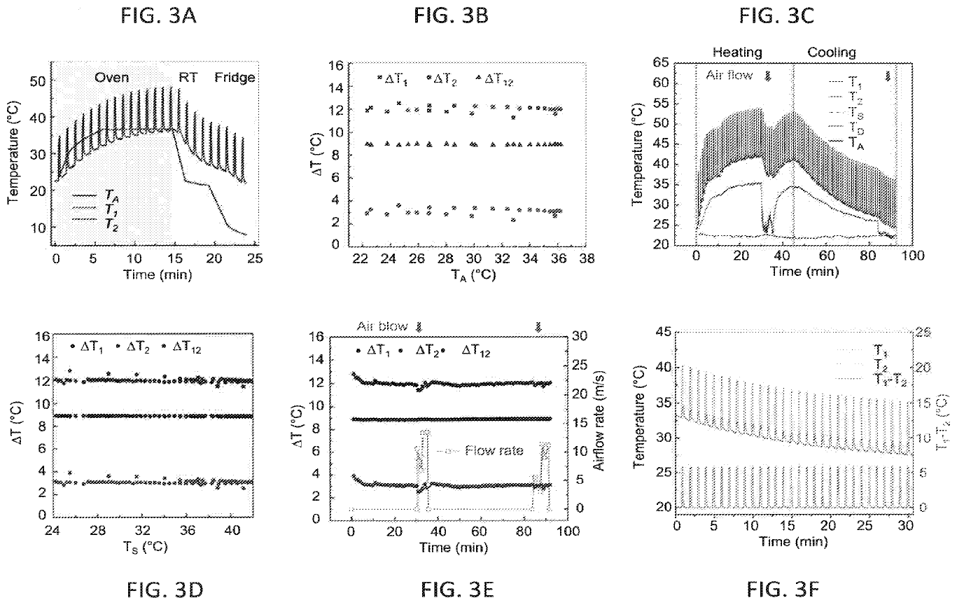

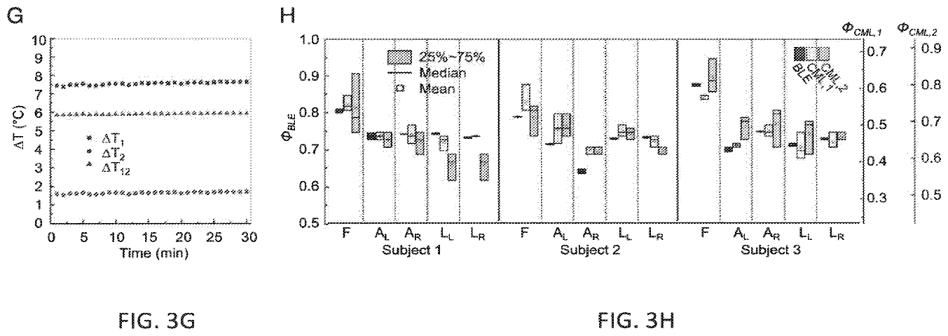

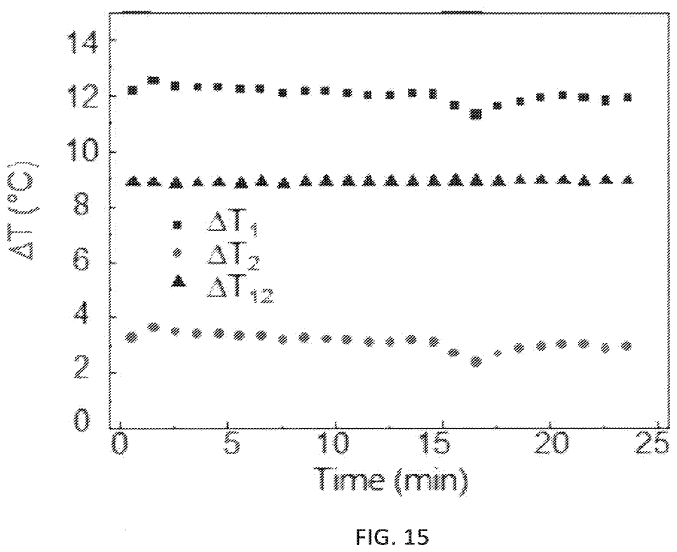

Experimental Studies: The use of ΔT₁₂, as measured with the two pairs of NTCs (NTC₁ and NTC₂) described previously, minimizes sensitivity to changes in skin temperature or variations in environmental conditions (air currents, ambient temperature, etc.). Demonstrations of the effects involve measurements of samples of S184 in an oven or refrigerator, or on a hot plate as the basis for varying the ambient temperature (T_{A}; FIGS. 3A-3B) and substrate temperature (T_{S}; FIGS. 3C-3D), respectively. A pneumatic valve provides control over the flow of air over the device, at rates between 0 and 13.6 m/s (FIGS. 3C and 3E). FIG. 3A shows wireless measurements of T₁ (blue) and T₂ (red) as a function of time, under conditions of varying ambient temperature, T_{A} (black), measured using a commercial thermometer (GM 1361; BENETECH). The value of T_{A} increases from 23.3 to 36.6° C. for 15 min in an oven (red background), and then decreases from 36.6 to 8.0° C. for an additional 9 min in a room temperature ambient (RT; white background) and subsequently in a refrigerator (blue background). Wireless measurements of ΔT₁ (black), ΔT₂ (red), and ΔT₁₂ (blue) as a function of T_{A} are shown in FIG. 3B. The SD for ΔT₁₂ across T_{A} from 8.0 to 36.6° C. is 0.03° C., roughly 10 times less than that associated with ΔT′ and ΔT₂ (0.27 and 0.29° C., respectively). The values of ΔT₁ and ΔT₂ fluctuate with abrupt increases and decreases of T_{A} at the moment the device enters and exits the oven, respectively (FIG. 15). FIG. 3C shows wireless measurements of T₁ (blue) and T₂ (red) as a function of time on a sample with varying temperature T_{S} (green) across a physiologically relevant range (from 24.2 to 41.0° C.; FIG. 3D), and with airflow rates of 0 to −13.6 m/s (FIG. 3E). T_{S} (green dashed line) is the base temperature measured from NTC₁ while the thermal actuator is off for 50 s every 1-min cycle. For measurements over a period of 90 min, T_{S} increases from 25.5 to 41.0° C. during the session labeled “heating” and decreases from 41.0 to 24.2° C. during the session labeled “cooling.” The surface temperature of the shell structure above the actuator of the device (TD; purple) changes accordingly (from 23.9 to 35.5° C. and then back to 21.8° C.), and T_{A} (black) is constant as −22.2±0.3° C. Varying the rate of airflow from the top (blue background) leads to abrupt changes in temperatures in the middle and toward the end of the heating and cooling process, respectively. Measurements of ΔT₁ (blue), ΔT₂ (red), and ΔT₁₂ (black) as a function of T_{S} (FIG. 3D) and as a function of time with airflow rates of 0 to −13.6 m/s (FIG. 3E) are in FIGS. 3D-3E, respectively. For cases of varying T_{S} and airflow rates, ΔT₁₂ exhibits an SD of 0.03° C., roughly six times less than that associated with ΔT₁ and ΔT₂ (0.17 and 0.17° C., respectively), and the SNR, SNR (dB)=20×log₁₀(ΔT_{12,mean}/ΔT_{12,SD})>50 dB, as listed in Table 1. The results indicate that the signals are typically >300 times larger than the noise. FIGS. 3 F and G shows the results of T₁ (blue), T₂ (red), and the difference (T₁−T₂; black), and ΔT₁ (black), ΔT₂ (red), and ΔT₁₂ (blue), respectively, for the case of immersing the device in cooled water (T_{S} from 33.1 to 27.3° C.). The biocompatible silicone packaging provides robust protection against water penetration such that the SDs of ΔT₁, ΔT₂, and ΔT₁₂ over measurement during a 30-min period are 0.07, 0.04, and 0.03° C., respectively. The hermetic sealing of the devices eliminates the effect of humidity of the surrounding environment on the circuit components.

TABLE 1
Signal-to-noise ratio (SNR) with different temperatures of
the testing substrate (Ts) for natural air convection and
for forced air flow at rates of 0~13.6 m/s from the top.

| | Time (min) | | | | | |
|---|---|---|---|---|---|---|
| | 0-10 | | | | | |
| | Abrupt | | 30-35 | | | 85-91 |
| | change in Ts | 10-30 | Airflow | 35-45 | 45-81 | Airflow |
| SNR (dB) of ΔT_{I} | 33 | 43 | 33 | 44 | 44 | 41 |
| SNR (dB) of ΔT₂ | 22 | 32 | 20 | 32 | 34 | 30 |
| SNR (dB) ΔT₁₂ | 58 | 59 | 66 | 61 | 54 | 66 |

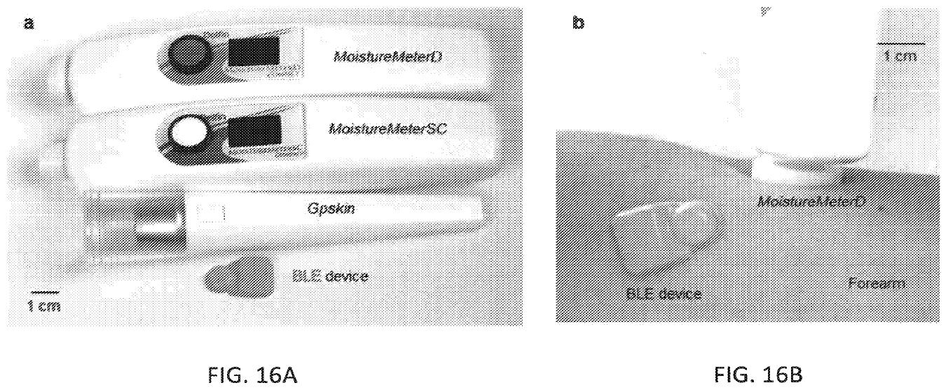

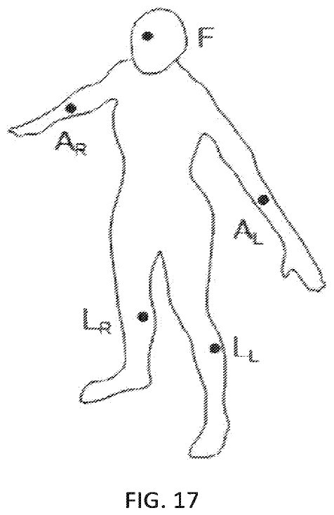

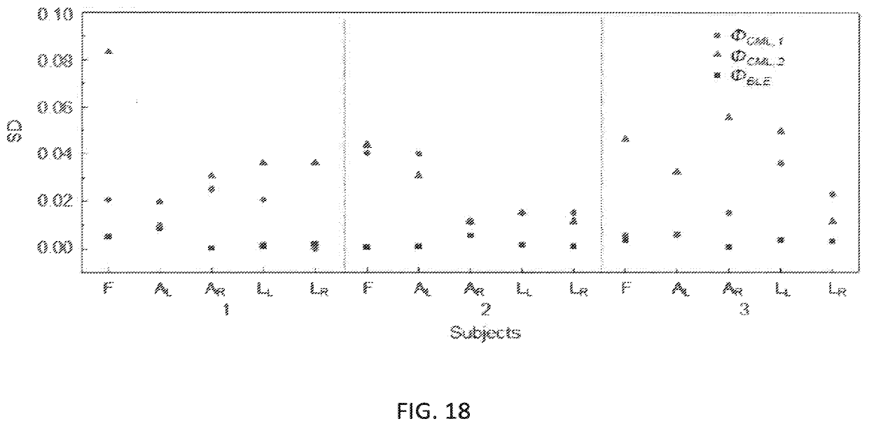

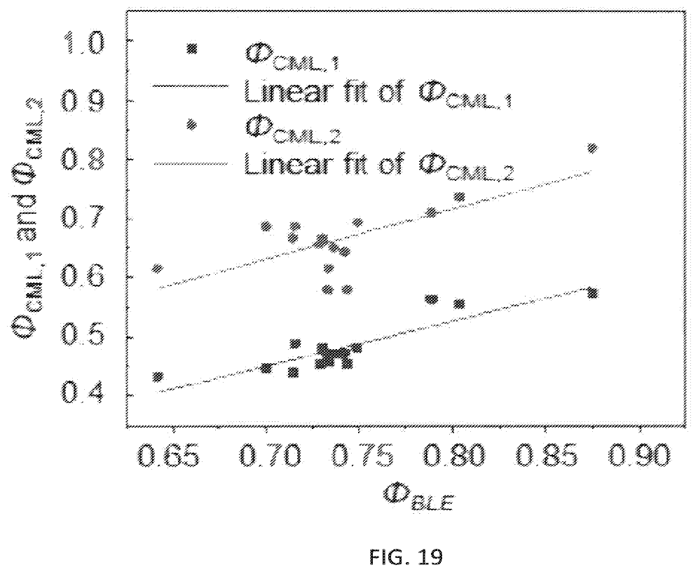

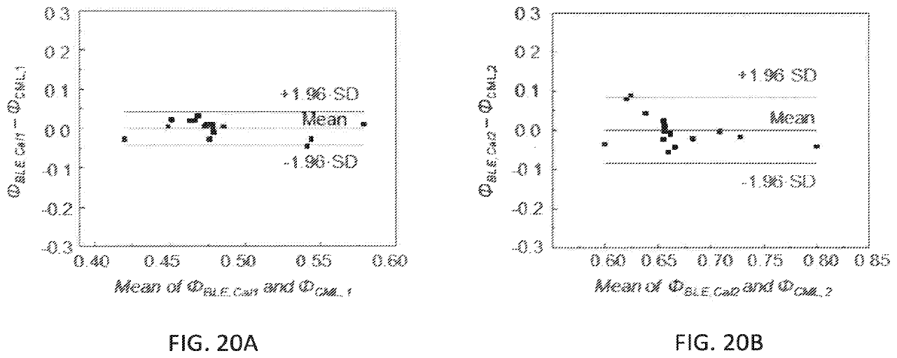

The devices can laminate gently, without applied pressure, onto the skin for determining Φ via measurements of ΔT₁₂, as described previously. The BLE interface supports wireless, long-range communication to smartphones, with user protocols that require almost no training or specialized skill (FIG. 3H). Basic tests involve measurements of Φ at a given body location by three different users from three different healthy subjects using the device according to embodiments of the invention (Φ_{BLE}) and commercial (CIVIL) devices for measuring tissue water content (Φ_{CML,1}; MoistureMeterD; Delfin Technologies) and skin surface hydration (Φ_{CML,2}; Gpskin; gpower) via measurements of skin dielectric properties (FIGS. 16A-16B). The measurement depth of the former is 500 to ˜2,500 μm, and that of the latter is 10 to ˜20 μm. The commercial devices require care by the user to hold the probe and manually apply a certain, fixed pressure against the skin for a few seconds for each measurement. FIG. 3H shows the results for Φ at five different body locations (FIG. 17): forehead (F), right arm (A_{R}), left arm (A_{L}), right leg (L_{R}), and left leg (L_{L}). User variability associated with Φ_{BLE}, Φ_{CML,1}, and Φ_{CML,2} at the same body location yields an average value of SDs of 0.00, 0.02, and 0.03, respectively. The SDs of Φ_{CML,1} and Φ_{CML,2} are the largest (0.04 and 0.09, respectively) on the forehead of subject 2 and 1, respectively, and that associated with Φ_{BLE} is constant (˜0.00) across these five body locations, each with a different curvature and rigidity (FIG. 18). The data show that Φ_{BLE} yields the most repeatable values of Φ. The results for Φ_{BLE} correlate with those from both Φ_{CML,1} and Φ_{CML,2} (FIG. 19). Linear fits indicate that Φ_{CML,1}=Φ_{BLE}×0.76−0.08 (R²=0.76), and Φ_{CML,2}=Φ_{BLE}×0.85+0.04 (R²=0.51). Bland-Altman plots (difference plots) shown in FIGS. 20A-20B show agreement between readings of Φ calibrated (Φ_{BLE,Cal1}=Φ_{BLE}×0.76−0.08, Φ_{BLE,Cal2}=Φ_{BLE}×0.85+0.04) to those from the commercial devices (Φ_{CML,1} and Φ_{CML,2}). The results show that Φ_{BLE} with calibration yields higher correlation with Φ_{CML,1} than with Φ_{CML,2}, likely due to the comparable sensing depths for Φ_{BLE} and Φ_{CML,1}.

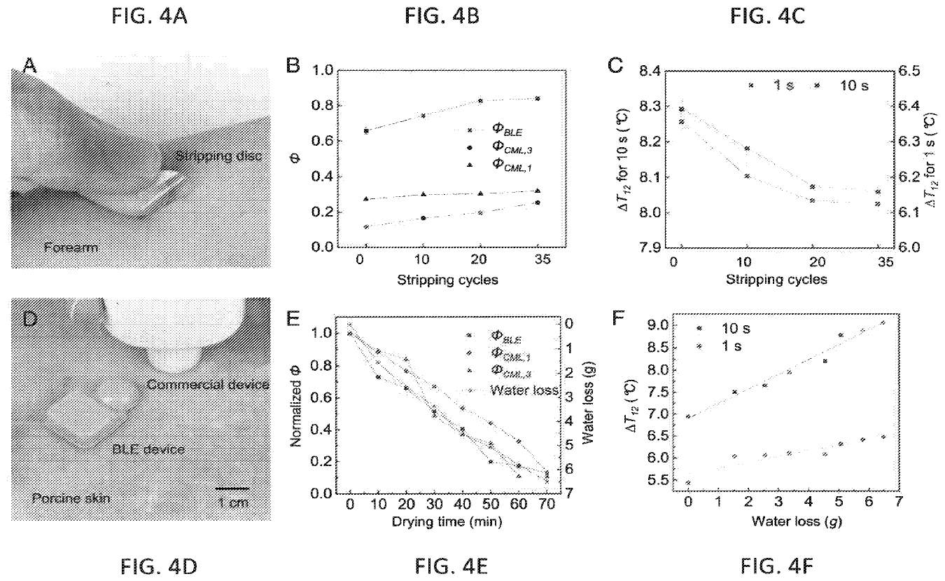

The measurement is sensitive to the presence and properties of the near surface layers of the skin, including the SC. As a demonstration, FIG. 4B shows measurements of Φ_{BLE} and Φ_{CML,1} (MoistureMeterD; Delfin Technologies) and SC hydration levels (Φ_{CML,3}) determined using a commercial device (MoistureMeterSC; Delfin Technologies; measurement depth of 40 um) as a function of the number of cycles of applying and removing an adhesive disk (D-Squame; CuDerm; FIG. 4A), as a simple and painless means to remove the SC. For increasing numbers of cycles, Φ_{BLE} increases in a systematic manner, as evidence of the sensitivity of the measurement to the SC. The data show strong correlations between the values of Φ_{BLE} and the SC hydration levels (Φ_{CML,3}), and number of stripping cycles, while the tissue water content (Φ_{CML,1}) is largely invariant. Values of ΔT₁₂ at short (t=1 s) and long (t=10 s) times as a function of stripping cycles, as in FIG. 4C, decrease by 3.6% and 2.8%, respectively, after 35 consecutive tape strips. The results indicate that the measurements at short times are more sensitive to the properties of the near surface layers of the skin, consistent with previous discussion of the thermal transport physics.

Studies of a sample of porcine skin (FIG. 4D) with different, known levels of hydration are in FIG. 4E. The changes in Φ normalized to the value of 1 shortly after placement of the sample in a food dehydrator (33° C.) exhibit strong correlation with independent measurements of the water loss of the sample (see Methods for details). Measurements of ΔT₁₂ at short (t=1 s) and long (t=10 s) times as a function of water loss are in FIG. 4F. The changes in ΔT₁₂ exhibit a positive correlation with water loss, as expected: ΔT₁₂ (10 s)=6.9+0.3×water loss (R²=0.97), and ΔT₁₂ (1 s)=5.6+0.1×water loss (R²=0.85).

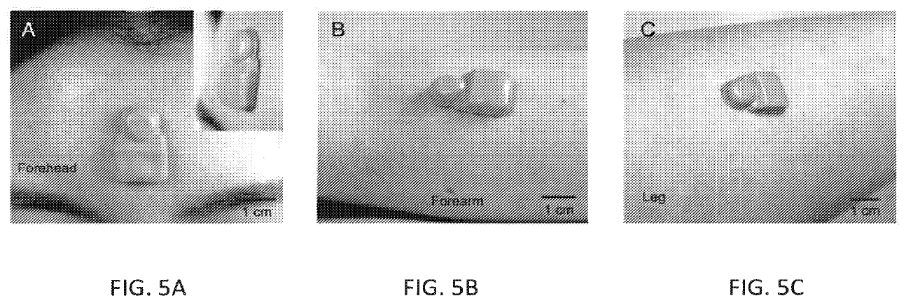

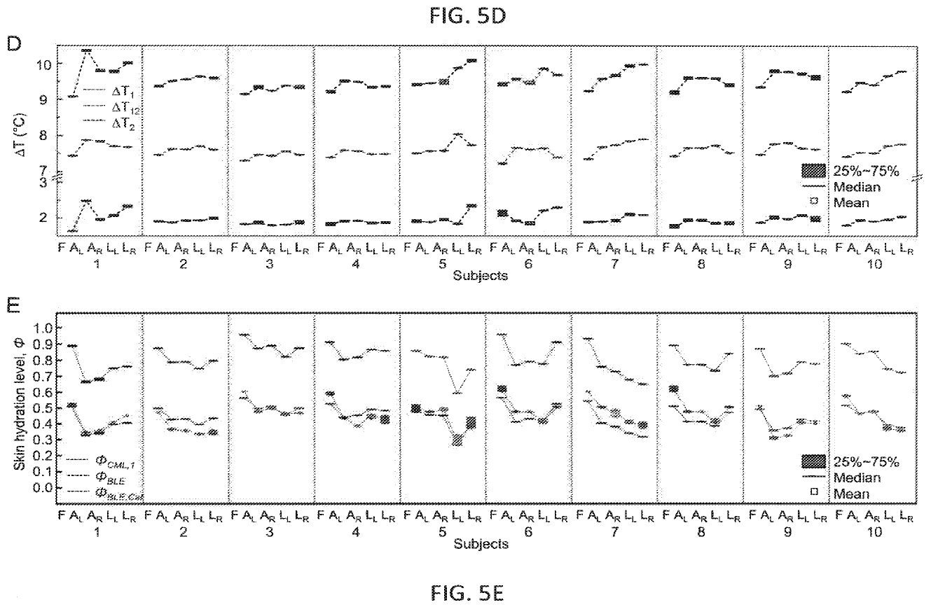

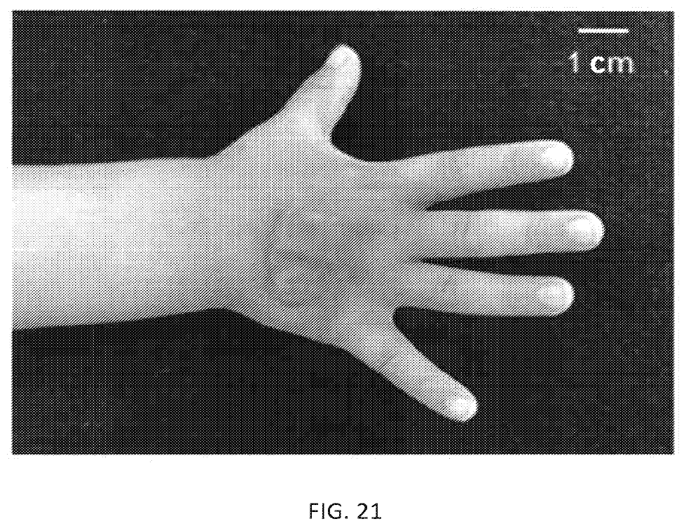

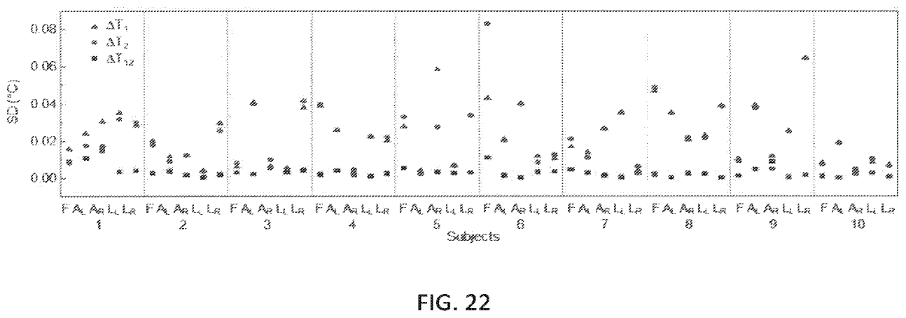

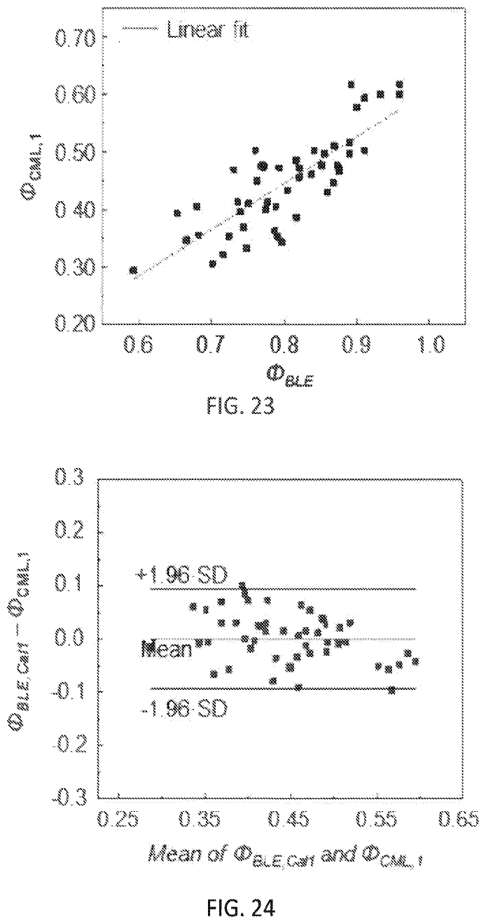

Human Subject Evaluations: These miniaturized, flexible platforms can be used on nearly any part of the human body, for adults and children (e.g., hand of a pediatric subject; FIG. 21) alike, including across highly curved or highly sensitive areas of the anatomy. FIGS. 5A-5C shows photographs of devices mounted on the forehead (FIG. 5A), forearm (FIG. 5B), and calf (FIG. 5C) of a human subject. The Inset in FIG. 5A features a tilted side view. Studies of hydration levels of the skin of 10 healthy volunteers involve evaluations at five different body locations (FIG. 17): forehead (F), right arm (A_{R}), left arm (A_{L}), right leg (L_{R}), and left leg (L_{L}). FIG. 5D shows ΔT₁, ΔT₂, and ΔT₁₂ over a 3-min measurement period from three female (subjects 1, 2, and 9; age range, 25 to 27) and seven male (subjects 3 to 8, 10; age range, 17 to 37) healthy volunteers (see Table 2) The results show that the forehead has the highest hydration level (the lowest values of ΔT₁, ΔT₂, and ΔT₁₂) across all subjects. The values of SDs for ΔT₁, ΔT₂, and ΔT₁₂ at each location for all subjects are less than ˜0.06, 0.08, and 0.01° C., respectively (FIG. 22). The data show that ΔT₁₂ yields the most consistent values of Φ, consistent with findings described in the previous sections. Comparisons of from values of ΔT₁₂ to those determined with a conventional handheld medical device (MoistureMeterD; Delfin Technologies; FIG. 16A) are in FIG. 5E. As before, Φ determined using the device introduced here (Φ_{BLE}) correlate strongly with those from the commercial device (Φ_{CML,1}; FIG. 23). Measurements of Φ with calibration coefficients (Φ_{BLE,Cal1}=Φ_{BLE}×0.80−0.20) correspond to Φ_{CML,1} with an average error (e) of e=|Φ_{BLE,Cal1}−Φ_{CML,1}|/Φ_{CML,1}=0.09 (FIG. 24).

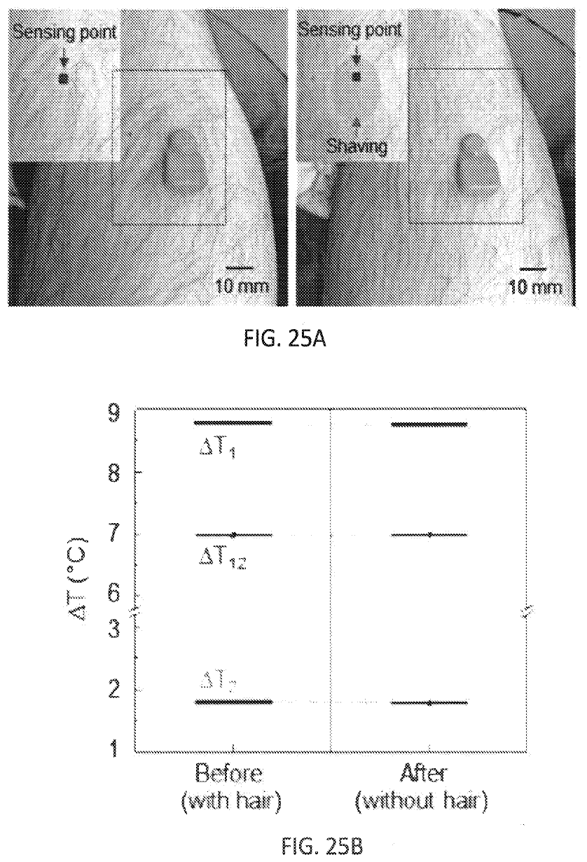

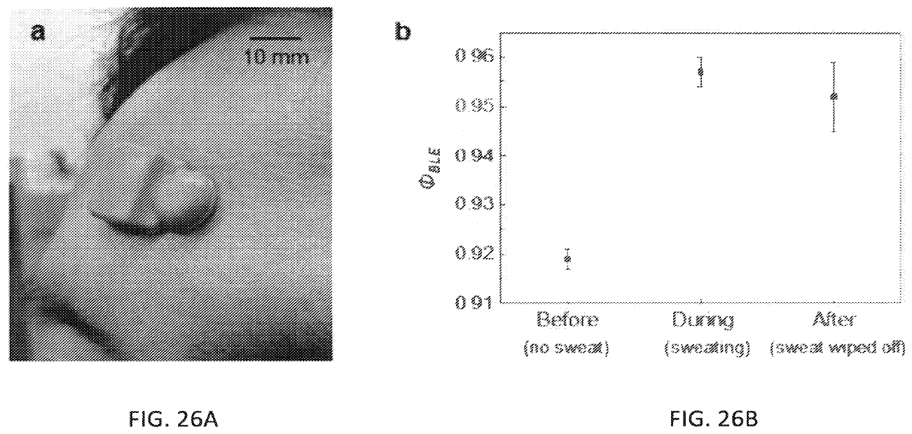

Additional experiments reveal the effect of hair-bearing skin on measurements of ΔT′, ΔT₂, and ΔT₁₂ (FIGS. 25A-25B). The mean±SD values of ΔT₁, ΔT₂, and ΔT₁₂ over 5-min measurements before and after shaving the skin are 8.76±0.03, 1.78±0.03, and 6.98±0.01° C. (before), and 8.78±0.03, 1.80±0.03, and 6.98±0.01° C. (after), respectively. The effect of perspiration on skin hydration levels before (no sweat), during (sweating), and after (sweat wiped off) a workout are shown in FIG. 26B. The sweat increases skin hydration levels (Φ_{BLE}) from 0.92 to 0.96 (no wiping)/0.94 (wiping), consistent with previous studies.

TABLE 2
Information of the 10 healthy normal subjects.

| Subject | | | | Fitzpatrick |
|---|---|---|---|---|
| number | Age | Sex | Ethnicity | Skin Type |
| 1 | 25 | F | Caucasian | I |
| 2 | 26 | F | Asian | II |
| 3 | 27 | M | Asian | II |
| 4 | 29 | M | Asian | II |
| 5 | 36 | M | Caucasian | I |
| 6 | 16 | M | Caucasian | I |
| 7 | 17 | M | Caucasian/Asian | I |
| 8 | 24 | M | Caucasian | I |
| 9 | 27 | F | Asian | II |
| 10 | 33 | M | Asian | II |

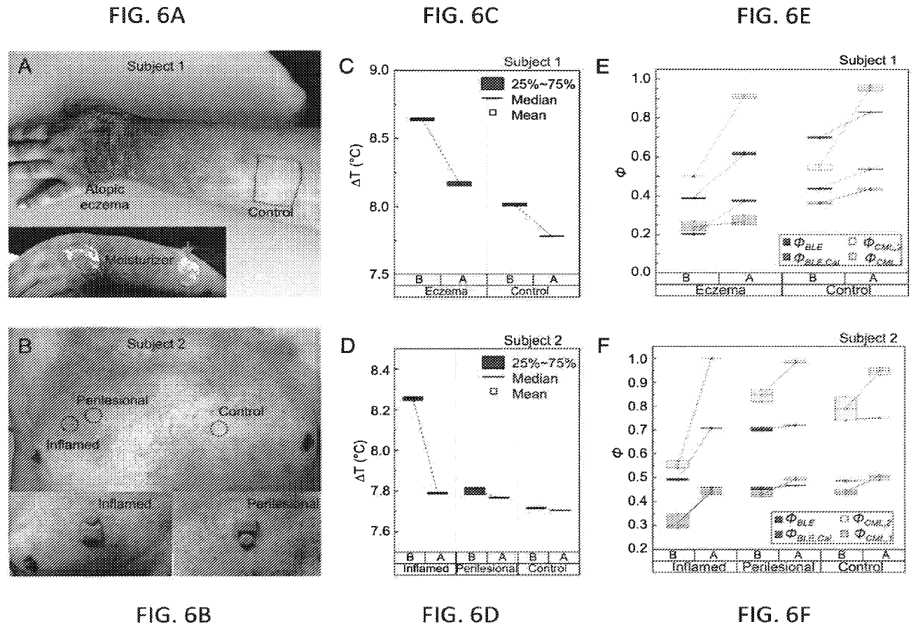

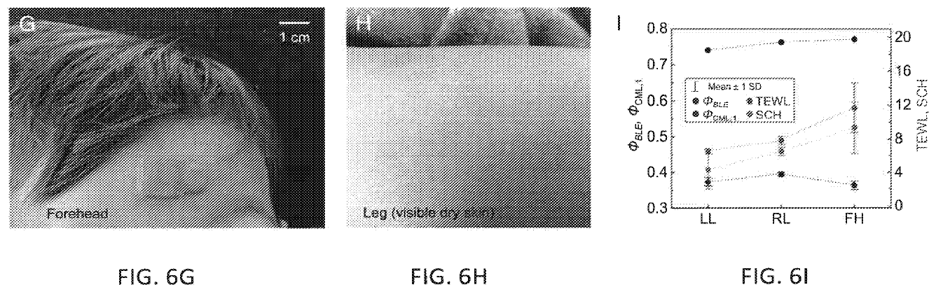

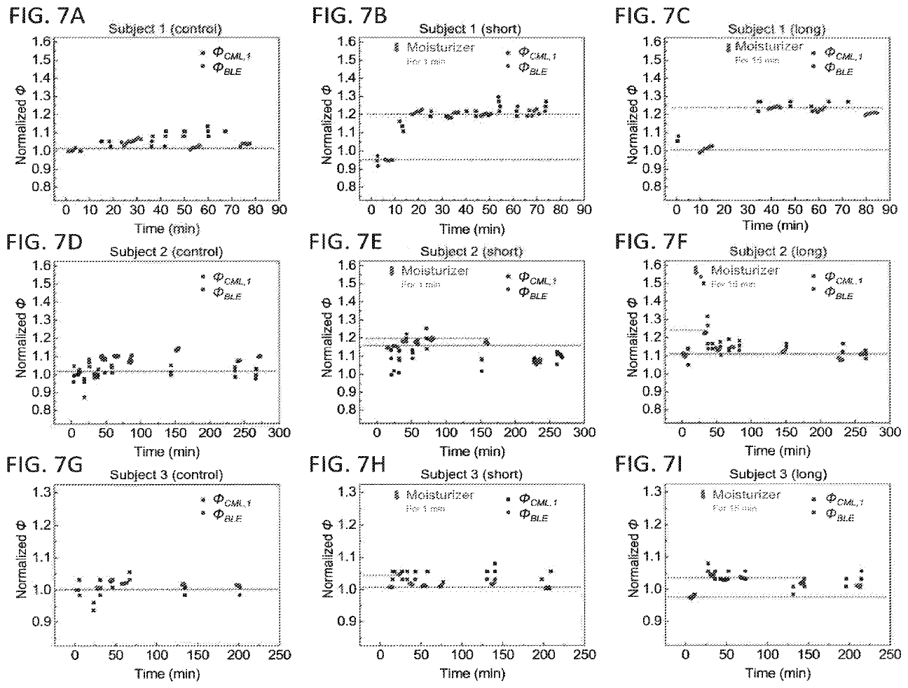

Evaluation of the Hydration Status of Pathological and Healthy Skin: Water originates from deep epidermal layers and gradually diffuses upward to hydrate cells of the SC, eventually leaving the skin via evaporation at volumes that are comparable to those lost on a daily basis by urination. Impaired skin increases this TEWL due to loss of barrier function from desiccation, infection, and mechanical stress. The following studies examine changes in the hydration status of disease-affected and clinically unaffected skin. Validation trials involve two patients with AD (subject 1 and 2, Tables 3-5), a toddler with visibly dry skin (FIGS. 6A-6I), and three young adults with healthy skin (FIGS. 7A-7I). FIGS. 6A-6B show the mounting locations on the back of the hand (atopic eczema), and the forearm (control) of subject 1 (FIG. 6A) and on the chest of subject 2 (inflamed, perilesional, and nonlesional skin from Left to Right; FIG. 6B). The Insets in FIGS. 6A-6B feature pictures of the forearm of subject 1 after application of moisturizer, and the platform mounted on inflamed (Left) and perilesional (Right) skin on the chest of subject 2, respectively.

TABLE 3
Information of the patients who participated
in the moisturizer study.

| Subject | | | | |
|---|---|---|---|---|
| number | Age | Sex | Ethnicity | Patholog |
| 1 | 22 | Female | African American | AD |
| 2 | 69 | Male | Latinx | AD |

TABLE 4
Φ measurements of a young adult patient with severe AD (subject 1).

| | | BLE | | MoistureMeterD | | Gpskin | | | |
|---|---|---|---|---|---|---|---|---|---|
| | | Φ_{BLE, Cal} | | Φ_{CML1} | | Φ_{CML, 2} | | | |
| | | mean | SD | mean | SD | mean | SD | TEWL | SCH |
| AD | before | 0.17 | 0.00 | 0.24 | 0.03 | 0.50 | 0.00 | 17 | 0 |
| | after | 0.32 | 0.00 | 0.36 | 0.01 | 0.91 | 0.01 | 33 | 41 |
| Control | before | 0.38 | 0.00 | 0.36 | 0.01 | 0.55 | 0.02 | 6 | 5 |
| | after | 0.46 | 0.00 | 0.44 | 0.01 | 0.96 | 0.02 | 21 | 46 |

TABLE 5
Φ measurements of an elderly patient with inflammatory AD (subject 2).

| | | BLE | | MoisinreMeterD | | Gpskin | | | |
|---|---|---|---|---|---|---|---|---|---|
| | | Φ_{BLE, Cal} | | Φ_{CML, 1} | | Φ_{CML, 2} | | | |
| | | mean | SD | mean | SD | mean | SD | TEWL | SCH |
| Inflamed | before | 0.29 | 0.00 | 0.31 | 0.03 | 0.55 | 0.02 | 22 | 5 |
| | after | 0.46 | 0.00 | 0.44 | 0.01 | 1.00 | 0.00 | 19 | 53 |
| Perilesional | before | 0.45 | 0.01 | 0.43 | 0.01 | 0.85 | 0.03 | 6 | 35 |
| | after | 0.47 | 0.00 | 0.49 | 0.01 | 0.98 | 0.01 | 4 | 48 |
| Control | before | 0.49 | 0.00 | 0.44 | 0.01 | 0.79 | 0.05 | 5 | 29 |
| | after | 0 49 | 0.00 | 0.51 | 0.01 | 0.95 | 0.02 | 2 | 45 |

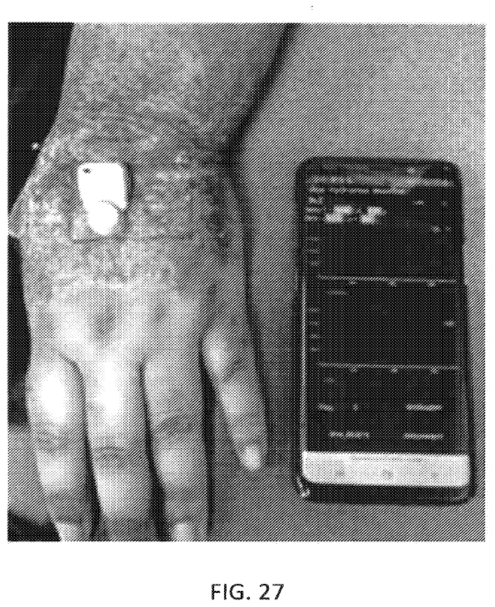

The optical image in FIG. 27 shows the platform on the atopic eczema of subject 1, next to a smartphone to collect/display/store the measurements. Results for ΔT₁₂ from subjects 1 and 2 are in FIGS. 6C-6D, respectively. Compared with healthy skin (control), lesional skin (eczema in FIG. 6C, inflammation in FIG. 6D) shows high values of ΔT₁₂ and a decrease in ΔT₁₂ before and 15 min after (B&A) applying moisturizer, respectively (see Methods for details). FIGS. 6E-6F show the values of Φ from ΔT₁₂ (Φ_{BLE}; red) and from MoistureMeterD (Φ_{CML,1}; sky blue) and Gpskin (Φ_{CML,2}; light green), for subjects 1 and 2, respectively. Compared with perilesional and nonlesional skin, atopic eczema and inflammation show low values of Φ_{BLE} (before) and an increase in Φ_{BLE} after application of moisturizer. The tissue water content (values of Φ_{CML,1}) correlates with calibrated values of Φ_{BLE} (Φ_{BLE,Cal}=Φ_{BLE}×0.76−0.08; pink) with an average error (e) of e=|Φ_{BLE, Cal}−Φ_{CML, 1}|/Φ_{CML,1}=0.09. The value of Φ_{CML,1} yields the largest SDs on lesions, lumpy and rigid area (0.03 on atopic eczema and inflamed skin, 0.01 on others), and an average value of SDs of 0.01, larger than that associated with Φ_{BLE,Cal} (0.00). Moisturizing the skin significantly increases the skin surface hydration level (values of Φ_{CML,2}) up to nearly 1 (0.91 on atopic eczema, and 1.00 on inflamed skin). FIGS. 6G-6H show the optical images of the device mounted on the forehead (FIG. 6G) and the leg (visibly dry skin determined by a dermatologist; FIG. 6H) of a toddler (male; age, 2). Measurements of Φ_{BLE} (blue), Φ_{CML,1} (black), TEWL (red), and SC hydration (SCH; green) on the left leg (L_{L}), right leg (R_{L}), and forehead (F_{H}) are in FIG. 6I. The values of TEWL and SCH measured using Gpskin device, and Φ_{BLE} are higher on the forehead where the hydration levels are expected to be higher than those on the leg.

Validation trials on three healthy adults (subject 1 with visible dry skin, and subjects 2 and 3 with not visible dry skin determined by a dermatologist) focus on observing variations in b after the application of moisturizer (˜4 h). The experimental protocol involves five steps: 1) wash the forearm with soap; 2) perform measurements at three different locations (“control,” “short,” and “long”) on the forearm; 3) apply a moisturizer (Extremely Dry Skin Rescue Lotion; Vaseline) on short and long areas, and wait for 1 min on short and 15 min for long; 4) wipe away excess moisturizer from the surface of the skin; and 5) repeat measurements at each location. The changes in Φ_{BLE} and Φ_{CML,1} normalized to each initial value at the control area are shown in FIGS. 7A-7I. The results indicate a strong correlation between Φ_{BLE} and Φ_{CML,1}. Compared to the initial values of Φ_{BLE} at the control area of subject 1 (FIG. 7A), the values of Φ_{BLE} at the short (FIG. 7B) and long (FIG. 7C) areas are 5% and 1% lower, respectively, at 0 min, and 20% and 23% higher immediately after the application of the moisturizer. At 80 min, Φ_{BLE} at the long area approaches to a value 20% higher than that at the control area. Compared to the initial values of Φ_{BLE} at the control area of a subject 2 (FIG. 7D), the values of Φ_{BLE} at the short (FIG. 7E) and long (FIG. 7F) areas are 20% and 23% higher after application of the moisturizer, and approach values 10% and 11% higher after ˜4 h. Compared to the initial values of Φ_{BLE} at the control area of a subject 3 (FIG. 7G), the values of Φ_{BLE} at the short (FIG. 7H) and long (FIG. 7I) areas are 5% and 4% higher after application of the moisture, and approach values 0% and 1% higher after ˜3 h. The increase in Φ_{BLE} after applying the moisturizer decreases with time.

Optimized Measurement Conditions: The optimization of the measurement conditions is very important in obtaining accurate/precise, and reproducible results. For instance, the best results are obtained when (1) the measurement is performed rapidly, to minimize effects of occlusion of natural processes of water vapor release from the skin due to the presence of the device, (2) very light or zero applied pressure is used during the measurement, to minimize perturbations to the skin (3) the adhesive is patterned such that it is present only across regions of the device adjacent to the sensor itself, to avoid exfoliation of the skin at the measurement site during peel back, for improved repeatability, (4) the temperature of the device is comparable to that of the skin, (5) skin itself is allowed to acclimate to the surrounding environment prior to the measurement.

### CONCLUSION

The soft, small, wireless platforms disclosed in the disclosure enable noninvasive, rapid monitoring of water content of healthy and diseased skin across a wide range of skin conditions, body locations, and subject backgrounds, with accuracy and precision superior to those of existing clinical or research-grade devices.

The combined use of an optimized, dual-sided TAS module with multiple, redundant measurement modalities supports repeatable, robust, user-independent measurements under various conditions relevant to practical use in both clinical and home settings. A BLE SoC interface to the phone allows for rapid data acquisition, suitable for operation with minimal training or specialized skills. Full-waveform fitting of the data captured using these systems to bilayer models of thermal transport yields hydration levels for both the epidermis and dermis. Evaluations of skin phantoms and partially hydrated porcine skin validate these measurement and analysis approaches. Pilot scale clinical studies with healthy and diseased subjects (n=19) illustrate a range of capabilities with clinical relevance. The results define the basis for versatile skin-interfaced devices that can support personalized and localized skin hydration strategies, with potential use as a diagnostic for skin disease states such as AD and XC, as a risk stratification tool for neonates at high risk for the development of AD, and as the basis for objective evaluation of the efficacy of topical medications and personal care product (e.g., topical moisturizers). Additional potential applications include monitoring thermoregulation processes and managing heat-related disorders.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various non-patent literature publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. J. Bolognia, J. Jorizza, J. Schaffer, Dermatology (Saunders, 2012).
[2]. J. du Plessis et al., International guidelines for the in vivo assessment of skin properties in non-clinical settings: Part 2. Transepidermal water loss and skin hydration. Skin Res. Technol. 19, 265-278 (2013).
[3]. L. DeSanti, Pathophysiology and current management of burn injury. Adv. Skin Wound Care 18, 323-334 (2005).
[4]. S. Nutten, Atopic dermatitis: Global epidemiology and risk factors. Ann. Nutr. Metab. 66 (suppl. 1), 8-16 (2015).
[5]. S. Mekic et al., Prevalence and determinants for xerosis cutis in the middle-aged and elderly population: A cross-sectional study. J. Am. Acad. Dermatol. 81, 963-969.e2 (2019).
[6]. M. Kelleher et al., Skin barrier dysfunction measured by transepidermal water loss at 2 days and 2 months predates and predicts atopic dermatitis at 1 year. J. Allergy Clin. Immunol. 135, 930-935.e1 (2015).
[7]. K. Horimukai et al., Transepidermal water loss measurement during infancy can predict the subsequent development of atopic dermatitis regardless of filaggrin mutations. Allergol. Int. 65, 103-108 (2016).
[8]. S. Kezic, J. B. Nielsen, Absorption of chemicals through compromised skin. Int. Arch. Occup. Environ. Health 82, 677-688 (2009).
[9]. F. L. Filon et al., In vitro absorption of metal powders through intact and damaged human skin. Toxicol. Vitro 23, 574-579 (2009).
[10]. R. Darlenski, S. Sassning, N. TSankov, J. W. Fluhr, Non-invasive in vivo methods for investigation of the skin barrier physical properties. Eur. J. Pharm. Biopharm. 72, 295-303 (2009).
[11]. B. Raynor, E. Ashbrenner, M. Garofalo, J. Cohen, F. Akin, The practical dynamics of transepidermal water loss (TEWL): Pharmacokinetic modeling and the limitations of closed-chamber evaporimetry. Skin Res. Tech. 10, 3 (2004).
[12]. J. C. Cohen et al., Comparison of closed chamber and open chamber evaporimetry. Skin Res. Technol. 15, 51-54 (2009).
[13]. B. Gabard, P. Treffel, “Transepidermal water loss” in Measuring the Skin, P. Agache, P. Humbert, Eds. (Springer, Berlin, Germany, 2004), pp. 553-564.
[14]. V. Rogiers; EEMCO Group, EEMCO guidance for the assessment of transepidermal water loss in cosmetic sciences. Skin Pharmacol. Appl. Skin Physiol. 14, 117-128 (2001).
[15]. J. Pinnagoda, R. A. Tupker, T. Agner, J. Serup, Guidelines for transepidermal water loss (TEWL) measurement. A report from the standardization group of the European Society of Contact Dermatitis. Contact Dermat. 22, 164-178 (1990).
[16]. S. Krishnan et al., Multimodal epidermal devices for hydration monitoring. Microsyst. Nanoeng., 3 (2017).
[17]. S. R. Madhvapathy et al., Epidermal thermal depth sensors: Epidermal electronic systems for measuring the thermal properties of human skin at depths of up to several millimeters. Adv. Funct. Mater. 28, 1870242 (2018).
[18]. M. Qassem, V. Kyriacoui, Review of modern techniques for the assessment of skin hydration. Cosmetics 6, 19 (2019).
[19]. S. E. Gustafsson, Transient plane source techniques for thermal conductivity and thermal diffusivity measurements of solid materials. Rev. Sci. Instrum. 62, 797-804 (1991).
[20]. T. Okabe et al., First-in-human clinical study of novel technique to diagnose malignant melanoma via thermal conductivity measurements. Sci. Rep. 9, 3853 (2019).
[21]. M. Guzmán-alonso, T. M. Cortazár, Water content at different skin depths and the influence of moisturizing formulations. Househ. Pers. Care Today 11, 35-40 (2016).
[22]. M. L. Clausen, H. C. Slotved, K. A. Krogfelt, T. Agner, Tape stripping technique for stratum corneum protein analysis. Sci. Rep. 6, 19918 (2016).
[23]. L. Lünnemann et al., Noninvasive monitoring of plant-based formulations on skin barrier properties in infants with dry skin and risk for atopic dermatitis. Int. J. Womens Dermatol. 4, 95-101 (2018).
[24]. T. Shiohara, Y. Sato, Y. Komatsu, Y. Ushigome, Y. Mizukawa, Sweat as an efficient natural moisturizer. Curr. Probl. Dermatol. 51, 30-41 (2016).
[25]. T. Shiohara, Y. Mizukawa, Y. Shimoda-Komatsu, Y. Aoyama, Sweat is a most efficient natural moisturizer providing protective immunity at points of allergen entry. Al-lergol. Int. 67, 442-447 (2018).
[26]. N. I. Dmitrieva, M. B. Burg, Increased insensible water loss contributes to aging related dehydration. PLoS One 6, e20691 (2011).
[27]. S. Purnamawati, N. Indrastuti, R. Danarti, T. Saefudin, The role of moisturizers in addressing various kinds of dermatitis: A review. Clin. Med. Res. 15, 75-87 (2017).
[28]. K. E. Crawford et al., Advanced approaches for quantitative characterization of thermal transport properties in soft materials using thin, conformable resistive sensors. Extreme Mech. Lett. 22, 27-35 (2018).
[29]. Madhvapathy, S. R. et al. Reliable, Low-Cost, Fully Integrated Hydration Sensors for Monitoring and Diagnosis of Inflammatory Skin Diseases in Any Environment. Under review.
[30]. Madhvapathy, S. R. et al. Epidermal Thermal Depth Sensors: Epidermal Electronic Systems for Measuring the Thermal Properties of Human Skin at Depths of up to Several Millimeters. Adv. Funct. Mater. 28, 1870242 (2018).
[31]. S. Krishnan et al., Multimodal epidermal devices for hydration monitoring. Microsystems & Nanoengineering 3, 17014 (2017).
[32]. E. Behrens, Thermal Conductivities of Composite Materials. J. Compos. Mater. 2, 2-17 (1968).

1. A hydration sensor, comprising:
a sensing module operably disposed on a target area of interest of skin of a living subject for detecting data associated with thermal properties of the skin; and
a wireless platform coupled with the sensing module for wireless data transmission between the sensing module and an external device.

2. The hydration sensor of claim 1, wherein the sensing module comprises:
a thermal actuator operably disposed on the target area of interest of the skin for heating the target area of interest thereof; and
a sensing circuit for simultaneously detecting a transient temperature change (ΔT) thereof to determine the thermal properties of the skin.

3. The hydration sensor of claim 2, wherein the thermal actuator and the sensing circuit are interconnected by serpentine traces to form a flexible structure that facilitates soft, intimate contact to the skin with robust mechanical and thermal coupling.

4. The hydration sensor of claim 2, wherein the thermal actuator comprises at least one resistor.

5. The hydration sensor of claim 4, wherein the thermal actuator comprises two or more of surface-mount thin film resistors, thick film resistors, through-hole resistors, and ultrathin-film metal resistors, coupled to each other in series.

6. The hydration sensor of claim 2, wherein the sensing circuit comprises one or more of negative temperature coefficient thermistors, positive temperature coefficient thermistors, resistance temperature detectors (RTD), and thermocouples.

7. The hydration sensor of claim 2, wherein the sensing circuit comprises a first pair of negative temperature coefficient thermistors (NTCs) arranged in a first Wheatstone bridge circuit.

8. The hydration sensor of claim 7,
wherein the first pair of NTCs is disposed on a layer different from the thermal actuator, and the first pair of NTCs is directly on the top of the thermal actuator; or
wherein the first pair of NTCs is disposed on a layer same as the thermal actuator, and each first NTC has a first distance from the thermal actuator.

9. The hydration sensor of claim 8, wherein the sensing circuit further comprises a second pair of NTCs arranged in a second Wheatstone bridge circuit serving to compensate for changes in an ambient temperature.

10. The hydration sensor of claim 9, wherein the second pair of NTCs is disposed on the same layer as the first pair of NTCs, and each second NTC is spatially apart from the first pair of NTCs and has a second distance from the thermal actuator.

11. The hydration sensor of claim 10, wherein the first and second distances are determined by the design requirement of depth sensitivity into the skin, and ranges from 10s of μm to a few mm.

12. The hydration sensor of claim 1, wherein the wireless platform comprises at least one of Wi-Fi, BLE, and NFC communication protocols.

13. The hydration sensor of claim 12, wherein the wireless platform comprises a Bluetooth low energy system on a chip (BLE SoC).

14. The hydration sensor of claim 13, wherein the BLE SoC comprises
a general-purpose input/output (GPIO) electrically coupled to the thermal actuator for providing a periodic current to activate the thermal actuator;
a differential amplifier (AMP) electrically coupled to the sensing circuit for amplifying a difference of bridge voltages;
an analog-to-digital converter (ADC) electrically coupled to the AMP to digitize output voltages of the AMP; and
a BLE radio configured to wirelessly transmit output signals of the ADC to the external device for processing to determine the hydration status of the skin, and receive data from the external device to activate a GPIO pin to provide the periodic current to the thermal actuator.

15. The hydration sensor of claim 14, wherein a digital on/off switch controlled through a custom application on the external device is adapted to enable BLE-connection and activation of the GPIO pin to source the periodic current into the thermal actuator.

16. The hydration sensor of claim 14, wherein the BLE SoC further comprises a microcontroller (μC) configured to activate the GPIO pin to source the periodic current into the thermal actuator.

17. The hydration sensor of claim 14, further comprising a power module for providing power to the sensing circuit and the wireless platform.

18. The hydration sensor of claim 17, wherein the power module comprises a battery.

19. The hydration sensor of claim 18, wherein the battery is a rechargeable battery operably rechargeable with wireless recharging.

20. The hydration sensor of claim 19, wherein the power module further comprises a wireless charging module for wirelessly charging the rechargeable battery.

21. The hydration sensor of claim 18, wherein the power module further comprises a failure prevention element including a short-circuit protection component or a circuit to avoid battery malfunction.

22. The hydration sensor of claim 1, further comprising a flexible substrate in the form of a flexible printed circuit board (fPCB) with circuit traces that interconnect the thermal actuator on a skin side, the NTCs on an air side, and the BLE SoC.

23. The hydration sensor of claim 22, wherein the flexible substrate is formed of a flexible material comprising polyimide (PI), or polyethylene terephthalate (PET).

24. The hydration sensor of claim 22, further comprising an encapsulating enclosure enclosing the thermal actuator, the wireless platform, the battery, and the fPCB.

25. The hydration sensor of claim 24, wherein the encapsulating enclosure comprises
a top layer for thermal, chemical and mechanical isolation of the hydration sensor from the environment; and
a bottom layer for providing a direct interface between the thermal actuator at the skin side of the fPCB and the skin.

26. The hydration sensor of claim 25, wherein the top layer is a shell-like top encapsulation layer including small air gaps for thermally, mechanically, and chemically insulating the critical sensing components.

27. The hydration sensor of claim 26, wherein the top layer is formed of a flexible material including silicone or silicone gel, low/high density polyethylene (LDPE/HDPE), polystyrene, Teflon®, and various other flexible polymers.

28. The hydration sensor of claim 25, wherein the bottom layer comprises a flexible adhesive for attaching the hydration sensor to the skin.

29. The hydration sensor of claim 28, wherein the bottom layer further comprises an ultrathin fabric of fiberglass/reinforcement material embedded in the flexible adhesive layer for enhancing the mechanical robustness of the hydration sensor.

30. The hydration sensor of claim 29, wherein the reinforcement material is flexible and has varying mesh density and thickness to lend tear resistance to the bottom layer.

31. The hydration sensor of claim 28, wherein the flexible adhesive layer is formed of silicone or silicone gel, or double-sided skin-safe adhesives, with the ratio of silicone and silicone gel being adjusted to co-optimize mechanical integrity and tackiness of the adhesive.

32. The hydration sensor of claim 1, wherein the external device is a smartphone, a tablet, a computer, or any electronic device with data reading/processing capability.

33. The hydration sensor of claim 2, wherein the thermal properties of the skin comprise thermal conductivity and thermal diffusivity of the skin that are related to water content of the skin, wherein the water content is a function of a skin depth.

34. The hydration sensor of claim 33, wherein the water content is determined from the measured temperature change ΔT vs. time t.

35. The hydration sensor of claim 33, wherein the water content and skin surface temperature are used to determine a normal state or a disease state of the skin.

36. The hydration sensor of claim 33, wherein the water content and skin surface temperature serve as quantitative metrics of an efficacy of a treatment of a skin disease, or other health and wellness products including skin moisturizers, lotions, and/or creams.

37. The hydration sensor of claim 1, being usable for monitoring the skin condition in a clinical setting and/or an at-home setting.

38. The hydration sensor of claim 1, being usable for delivering treatment, monitoring the effects, modulating the treatment protocol as necessary, and/or potentially predicting for flares based on quantitative, individualized measurements on specific lesion sites.

39. The hydration sensor of claim 1, being usable for monitoring water content of internal organs for various diseases where traditional monitoring techniques fail to offer continuous assessment of organ health.

40. The hydration sensor of claim 1, being usable for monitoring organs during organ transport for applications in organ transplant.

41. The hydration sensor of claim 1, being usable for applications to measure thermal conductivity, thermal diffusivity, heat capacity and other thermal properties of any material as a function of depth.

42. The hydration sensor of claim 1, being usable for applications to measure water content of any material surface as a function of depth, including hydrogels, plants (irrigation and agriculture applications), food preservation (dried food products, grains, fruits, meats), and/or concrete (industrial applications).

43. The hydration sensor of claim 1, being usable for monitoring composition of food/beverages, medicines/industrial chemicals.

44. The hydration sensor of claim 1, being re-usable and removal without irritation to the skin or damage to the hydration sensor.

45. The hydration sensor of claim 1, being compatible with alcohol-based cleaning wipes allowing for re-use across different users, without any damage to the hydration sensor or loss in efficacy of the hydration sensor adhesive.

46. The hydration sensor of claim 1, being sterilizable using alcohol, autoclave steam sterilization, and gas phase sterilization.

47. A method of fabricating a hydration sensor, comprising:
forming a flexible printed circuit board (fPCB) that interconnects electronics of the hydration sensor; and
forming an encapsulating enclosure enclosing the sensing module, the wireless platform and the fPCB, wherein the encapsulating enclosure comprises a top layer and a bottom layer.

48. The method of claim 47, wherein the fPCB is formed of a flexible material comprising polyimide (PI), polyethylene terephthalate (PET), or any one of them in combination with stiff PCB material including FR-4.

49. The method of claim 47, wherein the bottom layer comprises a layered structure of a first flexible layer, a second flexible layer, and a fabric of fiberglass/a reinforcement material embedded between the first flexible layer and the second flexible layer.

50. The method of claim 49, wherein each of the first flexible layer and the second flexible layer is formed of silicone or silicone gel, or double-sided skin-safe adhesives, with the ratio of the silicone and silicone gel being adjusted to co-optimize mechanical integrity and tackiness of the adhesive.

51. The method of claim 49, wherein the reinforcement material is flexible and has varying mesh density and thickness to lend tear resistance to the bottom layer.

52. The method of claim 49, wherein the bottom layer adheres to the f-PCB through use of silicone bonding material, epoxy, glue, or commercial adhesive.

53. The method of claim 47, wherein the top shell layer is formed of silicone or silicone gel, low/high density polyethylene (LDPE/HDPE), polystyrene, Teflon®, and various other flexible polymers.

54. The method of claim 47, wherein the electronics comprises:
a sensing module for detecting data associated with thermal properties of the skin; and
a wireless platform coupled with the sensing module for wireless data transmission between the sensing module and an external device.

55. The method of claim 54, wherein the sensing module comprises:
a thermal actuator for heating a target area of interest of the skin; and
a sensing circuit for simultaneously detecting a transient temperature change (ΔT) thereof to determine thermal properties of the skin.

56. The method of claim 54, wherein the wireless platform comprises at least one of Wi-Fi, BLE, and NFC communication protocols.

57. The method of claim 56, wherein the wireless platform comprises a Bluetooth low energy system on a chip (BLE SoC).

58. A method of monitoring and/or diagnosing a condition of a skin, comprising:
attaching a hydration sensor onto a target area of interest on the skin, wherein the hydration sensor comprises a thermal actuator, a sensing circuit, and a wireless platform for two-way data communication with an external device;
heating the target area of interest of the skin by the thermal actuator, simultaneously detecting data associated with thermal properties of the skin by the sensing circuit, and wirelessly transmitting the detected data, by the wireless platform, to the external device to determiner a transient temperature change (ΔT) thereof;
obtaining water content of the target area of interest of the skin from the temperature change (ΔT); and
determining a condition of the skin at the target area of interest based on the obtained water content.

59. The method of claim 58, wherein the water content comprises water content Φ_{E} of the epidermis and water content Φ_{D} of the dermis.

60. The method of claim 59, wherein the step of obtaining the water content comprises separately determination of Φ_{E} and Φ_{D} from the temperature change ΔT.

61. The method in claim 58, wherein the wireless platform transmits data through a wireless communication protocol including Near Field Communication (NFC), Wi-fi/Internet, Bluetooth/Bluetooth low energy (BLE), or GSM/Cellular Communication.

62. The method in claim 58, wherein said heating the target area of interest of the skin is formed by providing a periodic current to the thermal actuator.

63. The method in claim 62, wherein activation of the periodic current is controlled by a digital on/off switch through a custom application on the external device.

64. The method of claim 58, wherein said determining the condition of the skin at the target area of interest comprises comparing the obtained water content to a standard water content at the target area of interest so as to determine a normal state or a disease state of the skin.

65. The method of claim 64, wherein said determining the condition of the skin at the target area of interest comprises diagnosing a skin disease at the target area of interest based on wherein the obtained water content thereof.

66. The method of claim 65, wherein said determining the condition of the skin at the target area of interest comprises evaluating an efficacy of a treatment of the skin disease.

67. The method of claim 65, wherein said obtaining water content of the target area of interest of the skin, and said determining a condition of the skin are performed in the external device.

68. The method of claim 58, further comprising displaying the condition of the skin at the target area of interest in the external device.

69. The method of claim 58, further comprising forwarding the condition of the skin at the target area of interest to a professional and/or a service provider.

70. The method of claim 58, wherein the external device is a smartphone, a tablet, a computer, or any electronic device with data reading data reading/processing capability.

71. The method of claim 58, further comprising one or more steps of delivering treatment, monitoring the effects, modulating the treatment protocol as necessary, and/or potentially predicting for flares based on quantitative, individualized measurements on specific lesion sites.

72. The method of claim 58, being performed under one or more optimized measurement conditions of:
the measurement being performed rapidly, to minimize effects of occlusion of natural processes of water vapor release from the skin due to the presence of the hydration sensor;
very light or zero applied pressure being used during the measurement, to minimize perturbations to the skin;
the adhesive being patterned such that it is present only across regions of the hydration sensor device adjacent to the sensor itself, to avoid exfoliation of the skin at the measurement site during peel back, for improved repeatability;
the temperature of the hydration sensor being comparable to that of the skin; and
skin itself being allowed to acclimate to the surrounding environment prior to the measurement.
